(19) United States
(12) Patent Application Publication
(10) Pub. No.: US 2023/0357696 A1
(43) Pub. Date: Nov. 9, 2023

(54) HERMETICALLY OR ASEPTICALLY SEALED BIOREACTOR SYSTEM AND RELATED METHOD THEREOF

(71) Applicant: University of Virginia Patent Foundation, Charlottesville, VA (US)

(72) Inventors: George J. Christ, Crozet, VA (US); Gavin T. Garner, Charlottesville, VA (US)

(73) Assignee: University of Virginia Patent Foundation, Charlottesville, VA (US)

(21) Appl. No.: 18/014,726

(22) PCT Filed: May 28, 2001
(86) PCT No.: PCT/US2021/034940

§ 371 Date: Jan. 5, 2023

Related U.S. Application Data
(60) Provisional application No. 63/049,773, filed on Jul. 9, 2020.

(51) Int. Cl. C12M 1/12; C12M 1/00; C12M 3/00

(52) U.S. Cl. CPC C12M 37/04; C12M 25/14; C12M 23/52; C12M 23/46; C12M 29/00; C12M 23/38

(57) ABSTRACT

Disclosed herein are details of a hermetically or aseptically sealed bioreactor. The bioreactor comprises a bioreactor chamber, a membrane wall, a scaffold structure, a linear actuator, a linear transfer means, and a control system. Use of the bioreactor permits the inner scaffold structure to be moved and manipulated while still preserving a hermetic or aseptic seal inside the bioreactor chamber during operation.

### CROSS REFERENCE TO RELATED APPLICATIONS

The present application claims benefit of priority under 35 U.S.C § 119 (e) from U.S. Provisional Application Ser. No. 63/049,773, filed Jul. 9, 2020, entitled “Hermetically Sealed Bioreactor System and Related Method Thereto”; the disclosure of which is hereby incorporated by reference herein in its entirety.

The present application is related to International Patent Application Serial No. PCT/US2016/051948, entitled “BIOREACTOR AND RESEEDING CHAMBER SYSTEM AND RELATED METHODS THEREOF”, filed Sep. 15, 2016; Publication No. WO 2017/048961, Mar. 23, 2017; the disclosure of which is hereby incorporated by reference herein in its entirety.

The present application is related to International Patent Application Serial No. PCT/US2017/045299, entitled “BIOREACTOR CONTROLLER DEVICE AND RELATED METHOD THEREOF”, filed Aug. 3, 2017; Publication No. WO 2018/027033, Feb. 8, 2018; the disclosure of which is hereby incorporated by reference herein in its entirety.

The present application is related to International Patent Application Serial No. PCT/US2019/054744, entitled “MODULAR BIOFABRICATION PLATFORM FOR DIVERSE TISSUE ENGINEERING APPLICATIONS AND RELATED METHOD THEREOF”, filed Oct. 4, 2019; Publication No. WO 2020/072933, Apr. 9, 2020; the disclosure of which is hereby incorporated by reference herein in its entirety.

### FIELD OF INVENTION

The present disclosure relates generally to artificially manufacturing cells and tissues for use in medical procedures. More particularly, the present disclosure relates to using cyclic motion to stimulate cell growth and/or maturation in an enclosed bioreactor which can be sealed off from external contaminants.

### BACKGROUND OF THE INVENTION

Volumetric muscle loss (VML) is the traumatic or surgical loss of skeletal muscle that results in irrecoverable functional impairment, ranging from disfigurement to lifelong disability. Patients with VML cannot recover because their bodies cannot regenerate the lost muscle. Annually, at least 20 million automobile accidents result in traumatic injury to the extremities. In addition, 70% of battlefield injuries are musculoskeletal in nature. Congenital VML also plays a role. Each year, 4,440 babies are born with cleft lip with or without cleft palate and 2,650 babies are born with cleft palate.

Current treatment options include functional free muscle transfer to the injury site and physical therapy. The results of free muscle transfer are inconsistent and depend on the skill of the surgeon. Physical therapy has not been shown to significantly improve recovery after VML and does not restore skeletal muscle fibers. There are tissue engineering approaches currently under development to treat VML. However, there is significant room for therapeutic improvement in the timeliness and magnitude of functional recovery.

The tissue engineered muscle repair (TEMR) technology improves functional recovery following implantation in biologically relevant preclinical models of VML injury. The construct involves seeding muscle-derived cells (muscle progenitor cells (MPCs) onto a bladder acellularized scaffold (BAM), which is then preconditioned in a bioreactor under cyclical mechanical loading to produce a myogenic cellular phenotype for implantation into a VML rat model. For example, when implanted, this construct can restore function by approximately 70%.

An improved bioreactor environment will enable increased understanding of the mechanical and biological mechanisms in vitro that affect the speed and magnitude of functional recovery of skeletal musculature following implantation in vivo, for the long-term goal of developing a tissue engineered construct for effective, reproducible, and prolonged muscle repair. In an improved bioreactor environment, the impact of cyclic mechanical strain, which affects proliferation, gene expression, and synthesis of matrix proteins, and other cellular activities of tissues, can be better studied and evaluated. In this setting, bioreactors may be designed to apply cyclic mechanical stretch for various tissues, including muscle, tendon, cartilage, bone, and many tissue composites. Multiple cell seeding steps can also produce more differentiated myogenic phenotypes, and more consistent cell reseeding on the scaffold is viable. Longer term investigations also require improved biocontainment. However, there currently do not exist any commercially available or published systems that allow for the aseptic or hermetic maintenance of Tissue Engineered Medical Products (TEMPs) during biomechanical conditioning in a bioreactor for translational research purposes.

Stated another way, a major issue with the currently available bioreactor systems is potential contamination of the cells throughout the bioreactor preconditioning process. With other systems, there is a possibility of exposure to external contaminants (e.g., bacteria, yeast, or mold) during the incubation and preconditioning period which is necessary for tissue maturation, and thus, regenerative capacity upon implantation. There is also the possibility of leaks through clearance spaces within a hole in the side of the bioreactor through which a leadscrew or other direct mechanical coupling would pass. There is therefore a need in the art for a hermetically or aseptically sealed bioreactor to provide better modes for cell growth, maturation and transportation.

In contrast, however, with the current invention, these sources of contamination are mitigated or prevented as the chamber is sterilized and then aseptically or hermetically sealed after the cells are loaded into it and until the bioreactor chamber is received by the end user (e.g., a surgeon or researcher) immediately before use (e.g., implantation back into the patient or for experimental purposes).

Moreover, the present inventor asserts that one of the ultimate challenges for FDA-approvable biomanufacturing of Tissue Engineered Medical Products (TEMPs) is the development of a fully automated, closed-loop system—whereby cell/tissue growth and maturation, in the presence of preconditioning with biomechanical cues could be achieved under sterile/aseptic conditions in the same device that will be used for shipment to the end-user (and implantation in the patient). There is therefore a need in the art for a hermetically or aseptically sealed bioreactor to avoid disruption or risk of contamination during the tissue biomanufacturing process.

In contrast, however, with the current invention, in this setting, TEMPs requiring biomechanical cues prior to implantation could undergo tissue maturation in a single device, without further disruption or risk of contamination during the tissue biomanufacturing process and eventual shipping/delivery of the TEMP that occurs subsequent to cell seeding.

Referring to conventional practices, many available bioreactor designs are not high throughput, and the vertical designs do not allow reseeding of the cell scaffold. The conventional systems with allegedly high-throughput bioreactors for loading tendon explants do not allow for reseeding the cells on both sides of the scaffolds, which are secured vertically in the bioreactor. Conventional multi-specimen instruments are simply machines for testing, and are not designed to support cell growth. Bioreactors that require cells be seeded onto a culture plate on the bottom of the bioreactor do not allow for the use of a biological scaffold such as the BAM, which is necessary, as a cell delivery vehicle for implantation into an animal model. Multiple cell seeding steps have been shown to produce more differentiated myogenic phenotypes and thus potentially improve functional capacity, and while a design may be available for the consistent reseeding of cells on both sides of the scaffold, no design currently seals the chamber for the duration of the tissue maturation and/or delivery/shipping process to mitigate or prevent possible contamination, while maximizing tissue function and regenerative capacity.

### SUMMARY OF ASPECTS OF EMBODIMENTS OF THE PRESENT INVENTION

As mentioned above, the following patents, patent applications and patent application publications as listed below are related to aspects of embodiments of the present invention and are hereby incorporated by reference in their entirety herein. The bioreactor related systems, bioreactor related devices, bioreactor related components, bioreactor methods, bioreactor controllers, methods for bioreactor controllers, and non-transitory computer readable medium to execute a method for a bioreactor controller are considered part of the present invention, and may be employed within the context of the invention.
a. International Patent Application Serial No. PCT/US2016/051948, entitled “BIOREACTOR AND RESEEDING CHAMBER SYSTEM AND RELATED METHODS THEREOF”, filed Sep. 15, 2016; Publication No. WO 2017/048961, Mar. 23, 2017.
b. U.S. patent application Ser. No. 15/760,009, entitled “BIOREACTOR AND RESEEDING CHAMBER SYSTEM AND RELATED METHODS THEREOF”, filed Mar. 14, 2018; Publication No. US-2018-0265831-A1, Sep. 20, 2018.
c. International Patent Application Serial No. PCT/US2017/045299, entitled “BIOREACTOR CONTROLLER DEVICE AND RELATED METHOD THEREOF”, filed Aug. 3, 2017; Publication No. WO 2018/027033, Feb. 8, 2018.
d. U.S. patent application Ser. No. 16/322,691, entitled “BIOREACTOR CONTROLLER DEVICE AND RELATED METHOD THEREOF”, filed Feb. 1, 2019.
e. International Patent Application Serial No. PCT/US2019/054744, entitled “MODULAR BIOFABRICATION PLATFORM FOR DIVERSE TISSUE ENGINEERING APPLICATIONS AND RELATED METHOD THEREOF”, filed Oct. 4, 2019; Publication No. WO 2020/072933, Apr. 9, 2020.
f. U.S. patent application Ser. No. 17/282,117, entitled “MODULAR BIOFABRICATION PLATFORM FOR DIVERSE TISSUE ENGINEERING APPLICATIONS AND RELATED METHOD THEREOF”, filed Apr. 1, 2021.

An aspect of an embodiment of the present invention provides, but not limited thereto, a TEMR construct having an improved bioreactor environment that permits bioreactor preconditioning of a cell-seeded construct, under controlled sterile and/or aseptic conditions, in a closed system that permits gas and nutrient exchange, and furthermore, without disruption of the tissue maturation process until it is complete and the construct is ready for removal and testing or implantation. Such an environment will serve, but not limited thereto, two goals for advancing the field: 1) enhanced preclinical translational research for the development of an improved tissue engineering/regenerative medicine solution for VML along with a wide range of other clinical applications, and 2) accelerated clinical translation of Tissue Engineered Medical Products (TEMPs).

As such, an aspect of an embodiment of the present invention addresses arguably some of the most critical challenges of the TEMP biomanufacturing process, namely the cell/tissue disruption and biocontainment risks caused by the need to individually manually seed TEMPs as well as removing them from the “bioreactor conditioning” environment at the end of the cell/tissue maturation phase for placement in a second device for shipping, transport or transfer. An aspect of an embodiment of the present invention limits biocontainment during the tissue maturation phase of TEMP biomanufacturing—while maintaining the ability to provide biomechanical cues that are a necessary part of that tissue maturation process-which is required to improve regenerative capacity following implantation in a VML injury. Other critical design criteria of an aspect of an embodiment of the present invention may include, but are not be limited thereto, the following: 1) the incorporation of a flexible, modular design for the bioreactor and bioreactor chamber, 2) fine motor control of the actuator with no direct contact with the contents of the bioreactor chamber, 3) hermetically or aseptically sealed device capable of accommodating recirculating media to the tissue construct for provision of nutrients without opening said bioreactor chamber (e.g., providing a closed-loop system), 4) requirement for biocompatible (e.g., cell-friendly) materials for the manufacturing of said bioreactor that are easily sterilized (e.g., autoclavable). All of these requirements are met by one or more embodiments of the presently disclosed bioreactor system or at portions thereof. For example, but not limited thereto, the implementation of requirement #2, represents an efficient and elegant design incorporating a flexible membrane (such as a silicone or other flexible material sheet (or sheets)) that can avoid the contents of the bioreactor chamber from coming into direct contact with any parts or environment located outside the contents of the bioreactor chamber.

In an embodiment, during operation the bioreactor chamber may still need to be opened and closed whenever the cells are seeded. Thereafter, once the cells are seeded and the bioreactor chamber is closed and sealed accordingly, the mechanical stretching, as well as cell/tissue growth and maturation, and tissue transportation to the end user (e.g., surgeon or researcher) can occur without further disruption to the tissue engineered medical product (TEMP) in the sealed bioreactor. For instance, at the end of the cell/tissue growth and maturation phase in the bioreactor chamber there is no additional requirement for also placing the TEMP into a second device for the purpose of shipping or transporting. But rather, the grown/matured cells can remain in the bioreactor chamber which can then be shipped or transported. This feature simultaneously reduces the number of manual steps (thus yielding increased manufacturing automation and simplicity) as well as increasing safety/biocontainment of the construct.

In an embodiment, the bioreactor chamber can be shipped, transported, or transferred without other components or modules of the overall bioreactor attached, and without requiring opening the bioreactor chamber or otherwise breaching the hermetic or aseptic seal as part of the shipping, transporting, or transferring process. In contrast, the conventional approach required two separate entities such as requiring a “biomanufacturing device” and the “shipping (transport or transfer) device”.

An aspect of an embodiment of the present invention bioreactor may comprise, but not limited thereto, a bioreactor chamber, a membrane wall, a membrane mount structure, a scaffold structure, a lid structure, a linear actuator, a coupling mechanism, and a control system. The scaffold is double-sided so that cell cultures can be deposited on both sides of the scaffold (alternatively, the scaffold may be one-sided or a variety of other designs). The bioreactor chamber may be hermetically or aseptically sealed by the lid structure, in order to prevent contaminants and unfiltered gases or other materials from entering the bioreactor chamber. This example of a bioreactor may be made in such a way that portions of the reactor may be disassembled while still maintaining the hermetic or aseptic seal of the bioreactor chamber. Alternatively, it may also be made as a solid unit that is not able to be disassembled beyond such disassembly required for basic operation (such as attaching/removing lid or installing/removing the scaffold structure).

In an embodiment, the lid structure may be located on any wall of the bioreactor chamber. The lid structure may be sized to occupy only a portion of a wall of the bioreactor chamber or it may be sized to occupy the full area of a wall of the bioreactor chamber. In an embodiment more than one lid may be utilized.

It should be appreciated that while the chamber disclosed herein is related to a bioreactor, without departing from the scope of the invention, the chamber and its related operation with the membrane wall, scaffold structure, linear actuator, linear transfer means, and/or control system may be implemented for other applications unrelated to a bioreactor. Without wishing to be bound to any particular use for the chamber (whether applied to a bioreactor or non-bioreactor) other structures may be employed for the chamber (other than specifically the chamber) within the context of interfacing with the membrane wall (or other aspects of the present invention), such as, but not limited thereto, the following: housing, enclosure, box, container, casing, tank, compartment, cavity, room, building, vehicle, aircraft, watercraft, trunk, wall, partition, channel, roof, ceiling, duct, conduit, case, or pipe.

Further, in an embodiment, rather than the scaffold, other components may be employed requiring movement or displacement by the linear actuator or the like.

In an embodiment, instead of the scaffold structure, a variety of other structures or components may be used as desired or required for a given application and operation of the chamber or the alternative structure to the chamber, such as: housing, enclosure, box, container, casing, tank, compartment, cavity, room, building, vehicle, aircraft, watercraft, trunk, wall, partition, channel, roof, ceiling, duct, conduit, cylinder, case, or pipe.

Accordingly, another example of an aspect of an embodiment of the present invention is a bioreactor which contains a clamp, screw, or other fastening system which may be used to secure the scaffold structure in position to avoid unwanted movement. This clamp, screw, or other fastening system may use one or more clamps, screws, or other devices to secure the scaffold structure. The scaffold structure may be secured in place at various times and for various reasons. These reasons may include, but are not limited to, ensuring that components stay in position while assembling the bioreactor and associated components, aiding in coupling and decoupling of the coupling mechanism, and securing the device or parts of the device for shipment, transport, or transfer.

The present bioreactor may precondition TEMR constructs under static, cyclical, or otherwise variable mechanical stretch, while allowing for multiple iterations of cell seeding on the scaffold, with potentially multiple cell types (e.g., satellite cells, myoblasts, fibroblasts, endothelial cells, or other stem or progenitor cells). To avoid perturbing the system, an aspect of an embodiment of the present invention bioreactor features a removable construct that secures the cell seeding scaffold in place, and can be reinserted into a separate reseeding chamber or the same bioreactor chamber to seed the underside of the cell seeding scaffold. For example, but not limited to, a cell seeding brace may be used to secure a cell seeding scaffold in place and the cell seeding scaffold may be reinserted into a separate scaffold structure or bioreactor chamber. An aspect of an embodiment of the present invention bioreactor shall, among other things, improve functional outcomes in muscle regeneration to treat VML injuries.

Accordingly, another example of an aspect of an embodiment of the present invention is a bioreactor which uses a motor or other type of actuator in order to cause static, cyclical, or otherwise variable mechanical stretch and uses a magnetic coupling to transfer the stretch to the cell seeding scaffold. This magnet (or magnets) can be either permanent or electromagnets. The magnetic coupling will allow the motor or other actuator to be connected to the coupling in order to cause the transfer of motion while still keeping the bioreactor chamber hermetically or aseptically sealed.

An aspect of an embodiment of the present invention provides, among other things, a bioreactor comprising: a bioreactor chamber; a membrane wall disposed on said bioreactor chamber, wherein said bioreactor chamber and said membrane wall are configured to maintain sterility or sanitation within said bioreactor chamber while said bioreactor chamber is closed; a scaffold structure disposed inside said bioreactor chamber; a linear actuator disposed outside said bioreactor chamber; a linear transfer means for transferring linear motion between said linear actuator and said scaffold structure without breaching said membrane wall; and a control system in communication with said linear actuator configured to control the movement of said linear actuator.

An aspect of an embodiment of the present invention provides, among other things, a bioreactor device, said device comprising: a bioreactor chamber and a membrane wall disposed on said bioreactor chamber, wherein said bioreactor chamber is configured to hold a scaffold structure or other component; and said membrane wall is configured to allow transfer of linear motion to said scaffold structure or other component without breaching said membrane wall. Further, a complimentary system may further be provided wherein the system is configured to receive said bioreactor device, wherein said system comprises: a linear actuator disposed outside said bioreactor chamber, wherein said linear actuator is configured to provide said linear motion; and a linear transfer means for transferring linear motion between said linear actuator and said scaffold structure or said other component without breaching said membrane wall.

An aspect of an embodiment of the present invention provides, among other things, a bioreactor comprising: a bioreactor chamber; a membrane wall disposed on said bioreactor chamber; a scaffold structure disposed inside said bioreactor chamber; a linear actuator disposed outside said bioreactor chamber; a linear transfer means for transferring linear motion between said linear actuator and said scaffold structure without breaching said membrane wall; and a control system in communication with said linear actuator configured to control the movement of said linear actuator.

An aspect of an embodiment of the present invention provides, among other things, a bioreactor comprising: a bioreactor chamber; a membrane wall disposed on said bioreactor chamber, wherein said bioreactor chamber and said membrane wall are configured to maintain sterility or sanitation within said bioreactor chamber while said bioreactor chamber is closed; a scaffold structure disposed inside said bioreactor chamber; an actuator disposed outside said bioreactor chamber; a transfer means for transferring motion between said actuator and said scaffold structure without breaching said membrane wall; and a control system in communication with said actuator configured to control the movement of said actuator.

An aspect of an embodiment of the present invention provides, among other things, a hermetically or aseptically sealed bioreactor. The bioreactor comprises a bioreactor chamber, a membrane wall, a scaffold structure, a linear actuator (or non-linear actuator), a linear transfer means (or non-linear transfer means), and a control system. Use of the bioreactor permits the inner scaffold structure to be moved and manipulated while still preserving a hermetic or aseptic seal inside the bioreactor chamber during operation.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user/operator/customer/client or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the environmental, anatomical, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g., rat, dog, pig, monkey), etc. It should be appreciated that the subject may be any applicable human patient, for example.

As discussed herein, a “subject” may be any applicable human, animal, or other organism, living or dead, or other biological or molecular structure or chemical environment, and may relate to particular components of the subject, for instance specific tissues or fluids of a subject (e.g., human tissue in a particular area of the body of a living subject), which may be in a particular location of the subject, referred to herein as an “area of interest” or a “region of interest.”

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5). Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The invention itself, together with further objects and attendant advantages, will best be understood by reference to the following detailed description, taken in conjunction with the accompanying drawings.

These and other objects, along with advantages and features of various aspects of embodiments of the invention disclosed herein, will be made more apparent from the description, drawings and claims that follow.

### BRIEF DESCRIPTION OF THE DRAWINGS

The foregoing and other objects, features and advantages of the present invention, as well as the invention itself, will be more fully understood from the following description of preferred embodiments, when read together with the accompanying drawings.

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention. Note that some structures or elements may be omitted from some of the drawings and not shown for the sake of clarity. Some elements or structures may be omitted as well when not necessary to show the operation of the embodiments.

FIG. 1(A) schematically illustrates an embodiment of a bioreactor, a bioreactor chamber, a linear transfer means that transfers motion through or across a membrane wall, a linear actuator, a scaffold structure, and a control system.

FIG. 1(B) schematically illustrates an embodiment of a bioreactor system of which is made up of a bioreactor chamber, a scaffold structure, control system, and a linear actuator, wherein in this embodiment the linear transfer means is a coupling mechanism, comprising an inner coupling mechanism and an outer coupling mechanism, which is used to transfer linear motion through or across the membrane wall.

FIG. 2(A) schematically illustrates an embodiment of a bioreactor comprising a bioreactor chamber, a membrane wall, outer and inner coupling mechanisms on either side of the membrane wall, a scaffold structure, a linear actuator, and a control system.

FIG. 2(B) schematically illustrates an embodiment of the bioreactor of FIG. 2(A) with the outer coupling mechanism (e.g., linear actuator coupling) advanced linearly inward (toward the right as rendered), toward the bioreactor chamber, such that it rests against the outer surface of the membrane wall.

FIG. 2(C) schematically illustrates an embodiment of the bioreactor of FIG. 2(A) with the outer coupling mechanism, further advanced compared to FIG. 2(B), engaged against the membrane wall deforming the membrane wall so as to be in contact with the inner coupling mechanism.

FIG. 2(D) schematically illustrates an embodiment of the bioreactor of FIG. 2(C) having the inner and outer coupling mechanisms advanced linearly outward (toward the left as rendered) displacing the membrane wall so as to be stretched to the outer bounds of movement (or a specified outward position).

FIG. 2(E) schematically illustrates an embodiment of the bioreactor of FIG. 2(D) having the inner and outer coupling mechanisms advanced linearly inward (toward the right as rendered) displacing with the membrane wall so as to be stretched to the inner bounds of movement (or a specified inward position).

FIG. 3 schematically illustrates an embodiment of a bioreactor chamber with a lid structure, clamp screws, and membrane mount structure.

FIG. 4 schematically illustrates an embodiment of a membrane mount structure.

FIG. 5 schematically illustrates an embodiment of a membrane wall, having two surfaces.

FIG. 6 schematically illustrates an embodiment of a scaffold structure with cell seeding scaffolds and associated inner coupling mechanisms.

FIG. 7 schematically illustrates an embodiment of a cell seeding structure.

FIG. 8 schematically illustrates an embodiment of a cell seeding structure with a cell seeding brace.

FIG. 9 schematically illustrates a top view of an embodiment of a lid structure with lid ports.

FIG. 10 schematically illustrates a side view of the drawing of FIG. 9.

FIG. 11 schematically illustrates a front view of the drawing of FIG. 9.

FIG. 12 schematically illustrates top view an embodiment of an outer coupling mechanism and a linear actuator having a lead screw and motor.

FIG. 13 schematically illustrates a side view of the bioreactor with the section view location for FIG. 14 specified.

FIG. 14 schematically illustrates a top section view of the bioreactor in the initial state after the scaffold structure is installed, and neither the screws or clamps nor the coupling mechanism are engaged.

FIG. 15 schematically illustrates a side view of the bioreactor with the section view location for FIG. 16 specified.

FIG. 16 schematically illustrates a top section view of the bioreactor in a state following that shown in FIG. 14, where the clamps or screws are engaged in a way which prevents the scaffold structure from moving, and the coupling mechanism is not engaged.

FIG. 17 schematically illustrates a side view of the bioreactor with the section view location for FIG. 18 specified.

FIG. 18 schematically illustrates a top section view of the bioreactor in a state following that shown in FIG. 16, where the clamps or screws remain engaged and the coupling mechanism is engaged.

FIG. 19 schematically illustrates a side view of the bioreactor with the section view location for FIG. 20 specified.

FIG. 20 schematically illustrates a top section view of the bioreactor in a state following that shown in FIG. 18, where the clamps or screws are disengaged and the coupling mechanism remains engaged.

FIG. 21 schematically illustrates a side view of the bioreactor with the section view location for FIG. 22 specified.

FIG. 22 schematically illustrates a top section view of the bioreactor in a state following that shown in FIG. 20, where the clamps or screws remain disengaged, the coupling mechanism remains engaged, and the linear actuator is retracted causing one side of the scaffold structure to move and causing elongation of the cell seeding scaffold.

FIG. 23 schematically illustrates a side view of the bioreactor with the section view location for FIG. 24 specified.

FIG. 24 schematically illustrates a top section view of the bioreactor in a state following that shown in FIG. 22, where the linear actuator is returned to the starting or previous position in a non-elongated state (such as previously shown in FIG. 20) and the clamps or screws remain in a disengaged state and the coupling mechanism remains in an engaged state.

FIG. 25 schematically illustrates a side view of the bioreactor with the section view location for FIG. 26 specified.

FIG. 26 schematically illustrates a top section view of the bioreactor in a state following that shown in FIG. 24, where (after the linear actuator had already been returned to the starting position, for example) the clamps or screws are engaged in a way which prevents the scaffold structure from moving, and the coupling mechanism remains engaged.

FIG. 27 schematically illustrates a side view of the bioreactor with the section view location for FIG. 28 specified.

FIG. 28 schematically illustrates a top section view of the bioreactor in a state following that shown in FIG. 26, where the clamps or screws remain engaged, and the coupling mechanism is disengaged.

FIG. 29 schematically illustrates a side view of the bioreactor chamber disconnected from the linear actuator with the section view location for FIG. 30 specified.

FIG. 30 schematically illustrates a top section view of the bioreactor chamber disconnected from the linear actuator (not shown instant Figure), with the clamps or screws in the position where the scaffold structure is secured and prevented from moving.

FIG. 31 schematically illustrates an embodiment of a coupling mechanism 159 having the outer coupling mechanism 154 and inner coupling mechanism 152 wherein said outer coupling mechanism 154 and inner coupling mechanism 152 comprise an adhesive material 161, whereby the adhesive material 161 attach the inner and outer coupling mechanisms together across the membrane 110 (spanning the membrane, with the membrane there between) without causing a breach in the membrane 110.

FIG. 32 schematically illustrates an embodiment of a coupling mechanism 159 having the outer coupling mechanism 154 and inner coupling mechanism 152 wherein said outer coupling mechanism 154 and inner coupling mechanism 152 comprise one or more suction cups 163 or other type of negative pressure connector, whereby the one or both suction cups 163 attach the inner and outer coupling mechanisms together across the membrane 110 (spanning the membrane, with the membrane there between) without causing a breach in the membrane 110.

FIG. 33 schematically illustrates an embodiment of a coupling mechanism 159 having the outer coupling mechanism 154 and inner coupling mechanism 152 wherein said outer coupling mechanism 154 and inner coupling mechanism 152 comprise a buckle 165 that may include a frame 166 and prong 167 or other mechanical connector with a male and female component which attaches the inner and outer coupling mechanisms together across the membrane 110 (spanning the membrane, with the membrane there between) without causing a breach in the membrane 110.

FIG. 34 schematically illustrates an embodiment of a coupling mechanism 159 having the outer coupling mechanism 154 and inner coupling mechanism 152 wherein said outer coupling mechanism 154 and inner coupling mechanism 152 comprise having one or more ball and socket joints 181 that may include a ball 182 and socket 183 that couples the outer coupling mechanism 154 and inner coupling mechanism 152 and wherein either the ball or socket side of the connector is attached or molded into the membrane (and wherein the inner and outer coupling mechanisms are connected together across the membrane 110 (spanning the membrane, with the membrane there between) without causing a breach in the membrane).

FIG. 35 schematically illustrates an embodiment of a coupling mechanism 159 having the outer coupling mechanism 154 and inner coupling mechanism 152 wherein said outer coupling mechanism 154 and inner coupling mechanism 152 comprise one or more threaded rod, screw, or bolt connectors 185 coupled by attachment to a threaded receiving portion 186 or threaded socket attached or molded into the membrane 110 (and wherein the inner and outer coupling mechanisms are connected together across the membrane 110 (spanning the membrane, with the membrane there between) without causing a breach in the membrane).

FIG. 36 schematically illustrates an embodiment of a coupling mechanism 159 having the outer coupling mechanism 154 and inner coupling mechanism 152 wherein said outer coupling mechanism 154 and inner coupling mechanism 152 comprises one or more male-female 187 connectors that may include a male connector 188 and female connector 189 where female side of the connector is attached or molded into the membrane 110, and the corresponding male side of the connector is located on the outer and/or inner coupling mechanism (and wherein the inner and outer coupling mechanisms are connected together across the membrane 110 (spanning the membrane, with the membrane there between) without causing a breach in the membrane). In an alternative embodiment, the male side of the connector may be attached or molded into the membrane 110, and the corresponding female side of the connector is located on the outer and/or inner coupling mechanism.

### DETAILED DESCRIPTION OF EXEMPLARY EMBODIMENTS

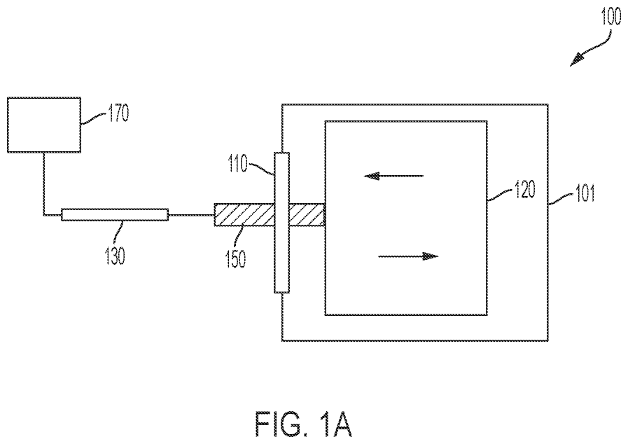

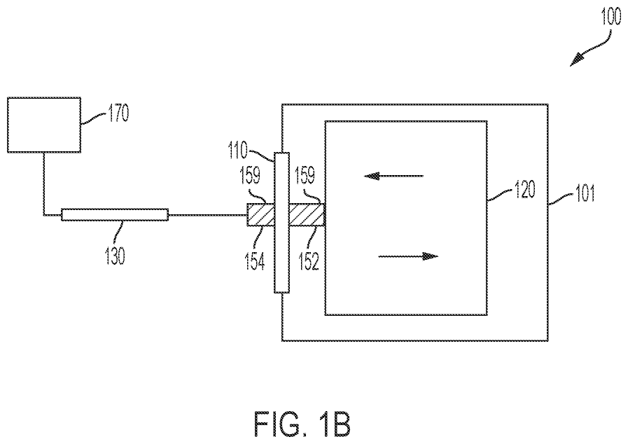

The present invention is described with reference to various embodiments of the invention. Throughout the description of the invention, reference is made to FIGS. 1-34. When referring to the figures, like structures and elements shown throughout are indicated with reference numerals. Note that some structures or elements may be omitted from some of the drawings and not shown for the sake of clarity. Some elements or structures may be omitted as well when not necessary to show the operation of the embodiments.

FIG. 1(A) schematically illustrates an embodiment of a bioreactor 100 which is made up of a bioreactor chamber 101, a linear transfer means 150 that transfers motion through or across a membrane wall 110, a linear actuator 130, a scaffold structure 120, and a control system 170.

FIG. 1(B) schematically illustrates an embodiment of a bioreactor system of which is made up of a bioreactor chamber 101, a scaffold structure 120, control system 170, and a linear actuator 130, wherein in this embodiment the linear transfer means is a coupling mechanism 159, comprising an inner coupling mechanism 152 (e.g., scaffold structure coupling) and an outer coupling mechanism 154 (e.g., linear actuator coupling), which is used to transfer linear motion through or across the membrane wall 110. The coupling mechanisms 159 may take the form of a coupling mechanism 159 configured to couple the linear actuator 130 to the scaffold structure 120 while maintaining the integrity of the membrane wall 110. In an embodiment, the control system 170 may be integrally formed with the linear actuator 130. In an embodiment, the control system 170 may be separately formed from the linear actuator 130. In an embodiment, the control system 170 and the linear actuator 130 may have varying subcomponents that have a mix of integrally formed and separately formed elements or functions.

In an embodiment, the control system 170 may be a mechanical, electrical, or electromechanical controller or processor. In an embodiment, this control system may contain, but is not limited to, one or more of a supervisory control and data acquisition system (SCADA), distributed control system (DCS), programmable logic controller (PLC), relay, cam timer, or drum sequencer. In an embodiment, the linear actuator 130 may be any one or more of the following: stepper motor, servomotor, rack and pinion, piston (e.g., pneumatic, magnetic or hydraulic type), crank and slider mechanism, solenoid, or leadscrew mechanism.

It should be noted that the control system 170 and linear actuator 130 do not permeate into the bioreactor chamber 101. For instance, the control system 170 and linear actuator 130 do not breach, occupy, penetrate, intrude upon, encroach nor invade the bioreactor chamber 101. When the bioreactor chamber 101 is sealed, no object will be able to invade into the bioreactor chamber 101. In an embodiment, the bioreactor chamber 101 may not contain any materials that may corrode or break down throughout the process (e.g., metals, non-sanitary materials), in order to limit contamination of the cells.

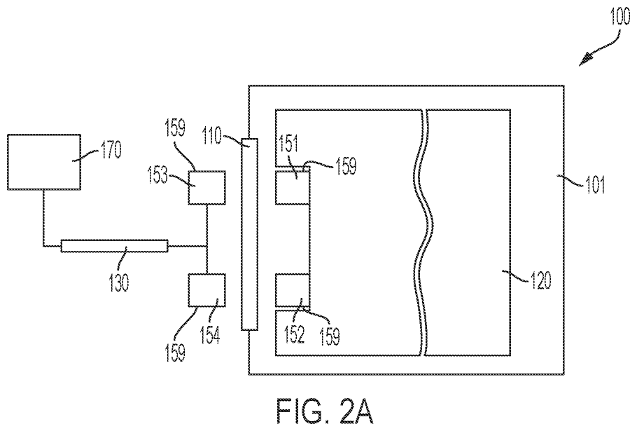

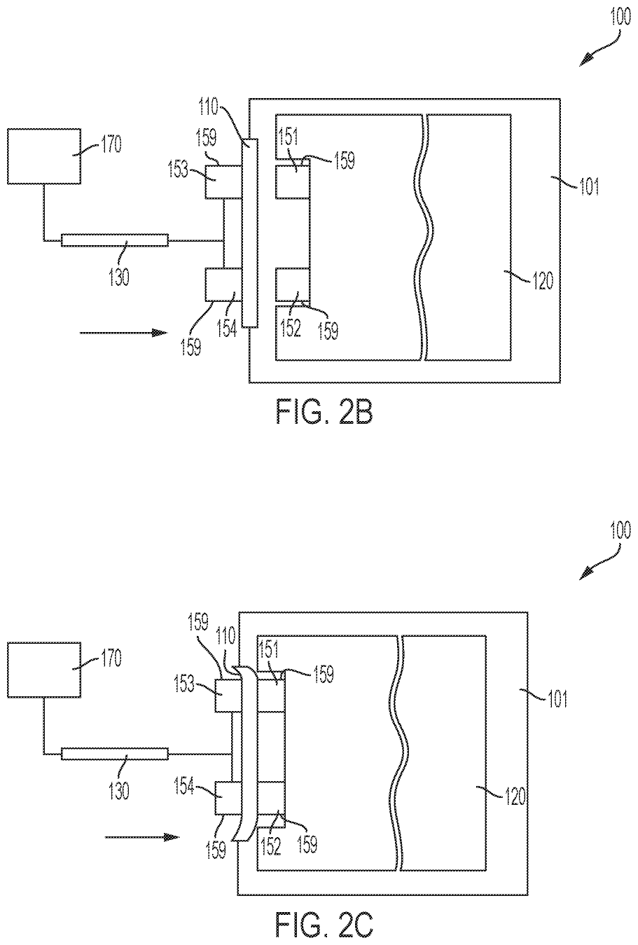

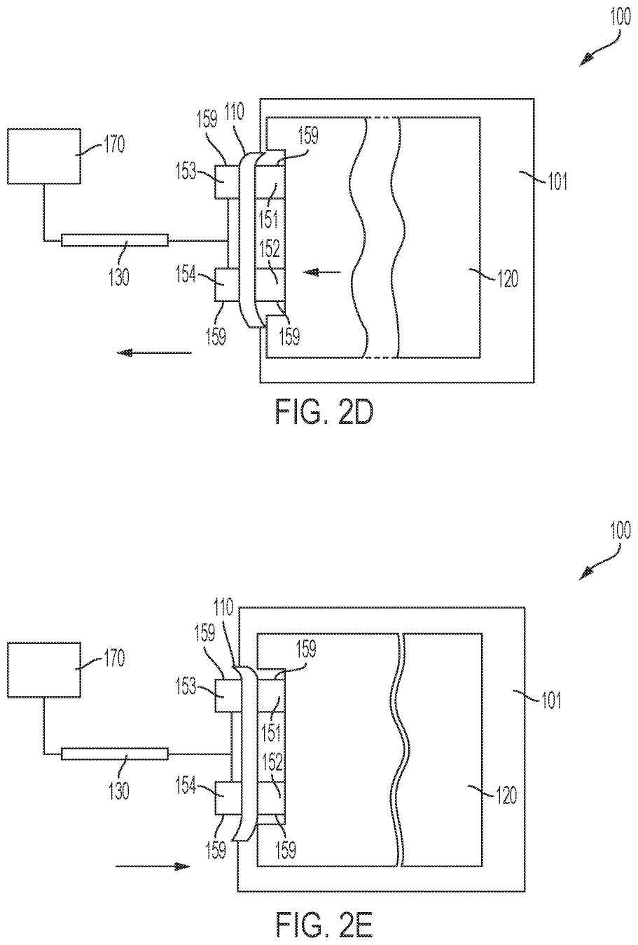

FIG. 2(A) schematically illustrates an embodiment of a bioreactor 100 comprising a bioreactor chamber 101, a membrane wall 110, control system 170, and a linear actuator 130, wherein the linear transfer means in this embodiment comprises a coupling mechanism 159 that includes outer coupling mechanisms 153, 154 (e.g., linear actuator couplings) and inner coupling mechanisms 151,152 (e.g., scaffold structure couplings) on either side of the membrane wall 110. Also shown is a scaffold structure 120 disposed in the bioreactor chamber 101. The coupling mechanisms 159 may take the form of a coupling mechanism 159 configured to couple the linear actuator 130 to the scaffold structure 120 while maintaining the integrity of the membrane wall 110. For example, but not limited thereto, a magnet may serve as either or both of the inner coupling mechanism 151, 152 (e.g., scaffold structure magnet) and outer coupling mechanism 153, 154 (e.g., linear actuator magnet).

FIGS. 2(B)-2(E) schematically illustrate a step-by-step process of the bioreactor representation of FIG. 2(A) engaging the coupling mechanism 159 and moving the scaffold structure 120 linearly along a path of motion desired for proper cell growth. It should be noted that although FIGS. 2(A)-2(E) contain two outer coupling mechanisms and two inner coupling mechanisms, this is not limiting on the design. In an embodiment, there may be one outer coupling mechanism and one inner coupling mechanism. In an embodiment, there may be three or more (or any plurality) outer coupling mechanisms and three or more (or any plurality) inner coupling mechanisms. In an embodiment, there may be any combination of one or more outer coupling mechanisms with one or more inner coupling mechanisms. In an embodiment, there may be any combination of one or more coupling mechanisms on one side of the membrane but not a corresponding coupling mechanism on the opposing side of the membrane (or a fewer number of coupling mechanisms on the opposing side of the membrane). Moreover, any of the types or styles of individual coupling mechanisms provided in this disclosure may be intermixed or matched with other (dissimilar) types or styles of individual coupling mechanisms.

Various aspects of embodiment of the invention may contain one or more outer and inner coupling mechanisms and a variety of types. For example, but not limited thereto, as shown later at least in part in FIGS. 1(B), 2A)-2(E), 13-36, these coupling mechanisms 159 may take the form of a coupling mechanism 159 configured to couple the linear actuator 130 to the scaffold structure 120 while maintaining the integrity of the membrane wall 110, wherein the coupling mechanism 159 can be in the form of, but is not limited to, one or more of any combination of the following:
a) an adhesive material 161 on either or both of the inner coupling mechanism 152 and outer coupling mechanism 154;
b) a magnet serving as either or both of the inner coupling mechanism 151, 152 and outer coupling mechanism 153, 154;
c) a magnet or ferromagnetic material serving as either or both of the inner coupling mechanisms 151, 152 and either or both of the outer coupling mechanisms 153, 154;
d) a suction cup 163 as part of either or both the inner coupling mechanism 152 and outer coupling mechanism 154;
e) a ball and socket 181 as part of either or both of the inner coupling mechanism 152 and outer coupling mechanism 154;
f) a screw/bolt/threaded rod 185 and threaded socket 186 as part of either or both the inner coupling mechanism 152 and outer coupling mechanism 154; or
g) a male and female connector 187, wherein the male connector 188 is opposite the female connector 189 and can be part of either the outer coupling mechanism 154 or inner coupling mechanism 152.

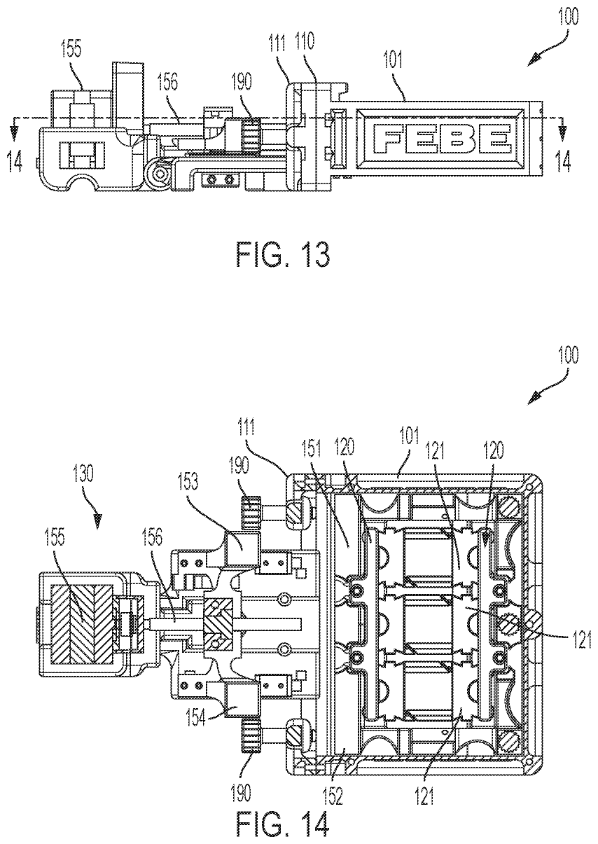

For the sake of simplifying some of the illustrations, FIGS. 13-30, are depicted without the lid 140 disposed thereon. During normal operations, in an embodiment the bioreactor chamber 101 may include a lid 140 that may switch from being in either open or closed positions. During normal operations, in an embodiment the bioreactor chamber 101 may include a lid 140 that may disposed on top of the perimeter walls of the bioreactor chamber 101 to close the bioreactor chamber 101 or the lid 140 may be removed therefrom so as to open the bioreactor chamber 101. In various embodiments, it may be designed such that the lid or the like may be oriented on the top the bioreactor chamber 101 or may be oriented so as to take the place of any of the side walls or the bottom of the bioreactor chamber 101. Moreover, an example of the bioreactor chamber being closed is when the lid 140 is in a closed position or disposed on top of the bioreactor chamber 101 thereby closing off the top of bioreactor chamber 101, and whereby the bioreactor is formed by at least one side wall and a membrane wall 110.

In an embodiment the membrane wall 110 may make up an entire side wall (or top or bottom) of the bioreactor chamber 101 or only a portion of a side wall (or top or bottom) of the bioreactor chamber 101. Said differently, the area of the membrane wall 110 may be substantially equal to the area of one of the side walls (or bottom or top) of the bioreactor chamber 101 or may be substantially less (or slightly less) than the area of one of the side walls (or bottom or top) of the bioreactor chamber 101. In an embodiment the area of the membrane wall 110 may be greater than the area of an entire side wall (or top or bottom) of the bioreactor chamber 101. In an embodiment the membrane wall 110 may be integrally formed with side wall (or top or bottom) of the bioreactor chamber 101 while still maintaining the seal and characteristics disclosed herein. In an embodiment the membrane wall 110 may be separately formed from side wall (or top or bottom) of the bioreactor chamber 101 while still maintaining the seal and characteristics disclosed herein.

In an embodiment the lid 140 may be only a flap, door, or window that can opened and closed. In an embodiment the lid (or flap, door, or window) may have an area less than the area of the top of the bioreactor chamber 101, a side wall of the bioreactor chamber, or bottom of the bioreactor chamber. Said differently, the lid (or flap, door, or window) may be only a portion of the area of the top of the bioreactor chamber 101, a side wall of the bioreactor chamber, or the bottom of the bioreactor chamber.

In an embodiment the lid 140 (or flap, door, or window) may have an area substantially equal to the area of the top of the bioreactor chamber 101, a side wall of the bioreactor chamber, or the bottom of the bioreactor chamber. In an embodiment the area of the lid 140 may be greater than the area of an entire side wall (or top or bottom) of the bioreactor chamber 101.

FIG. 2(A) schematically illustrates an embodiment of a bioreactor 100 comprising a bioreactor chamber 101, a membrane wall 110, a coupling mechanism 159 comprising outer coupling 153, 154 (e.g., linear actuator coupling) and inner couplings 151, 152 (scaffold structure coupling) on either side of the membrane wall 110, a scaffold structure 120, a linear actuator 130, and a control system 170.

FIG. 2(B) schematically illustrates an embodiment of the bioreactor 100 of FIG. 2(A) with the outer couplings 153, 154 (e.g., linear actuator coupling) advanced linearly inward (toward the right as rendered and generally reflected by the arrow), and toward the bioreactor chamber, such that it rests against the outer surface of the membrane wall 110.

FIG. 2(C) schematically illustrates an embodiment of the bioreactor 100 of FIG. 2(A) with the outer couplings 153, 154 (e.g., linear actuator coupling), further advanced compared to FIG. 2(B), engaged against the membrane wall deforming the membrane wall inward (toward the right as rendered and generally reflected by the arrow) so as to be in contact with the inner couplings 151, 152 (e.g., scaffold structure couplings).

FIG. 2(D) schematically illustrates an embodiment of the bioreactor 100 of FIG. 2(C) having the inner couplings 151, 152 (e.g., scaffold structure couplings) and outer couplings 153, 154 (e.g., linear actuator coupling) advanced linearly outward (toward the left as rendered and generally reflected by the arrow) displacing the membrane wall 110 so as to be stretched to the outer bounds of movement (or a specified outward position).

FIG. 2(E) schematically illustrates an embodiment of the bioreactor of FIG. 2(D) having the inner coupling 151, 152 (e.g., scaffold structure couplings) and outer couplings 153, 154 advanced linearly inward (toward the right as rendered and generally reflected by the arrow) displacing with the membrane wall 110 so as to be stretched to the inner bounds of movement (or a specified inward position). In an embodiment, the inner couplings 151, 152 (e.g., scaffold structure couplings) and outer couplings 153, 154 (e.g., linear actuator coupling) may be advanced linearly inward or outward to any most outer bound or inner bound position or any specified position subsumed there between for any specified number of times or repetitions at any specified speed or duration.

While an embodiment in FIGS. 2(A)-2(E) indicates the linear advancement inward (forward) or outward (backward) as being toward the right or left as rendered and generally reflected by the arrows (e.g., one-directional axis), respectively, it should be appreciated that alternative advancement patterns, paths or axes are considered part of the present invention, and may be employed within the context of the invention. For example, but not limited thereto, an advancement may be in any x, y, or z axis (or a combination thereof). For example, but not limited thereto, an advancement may be in an oval or elliptical pattern with any such oval or elliptical pattern lying in any possible plane in the x, y, or z axis (or a combination thereof). For example, but not limited thereto, an advancement may be along the entire continual geometric spectrum of manipulation of x, y and/or z axes to provide and meet structural demands and operational requirements of the bioreactor and related bioreactor process.

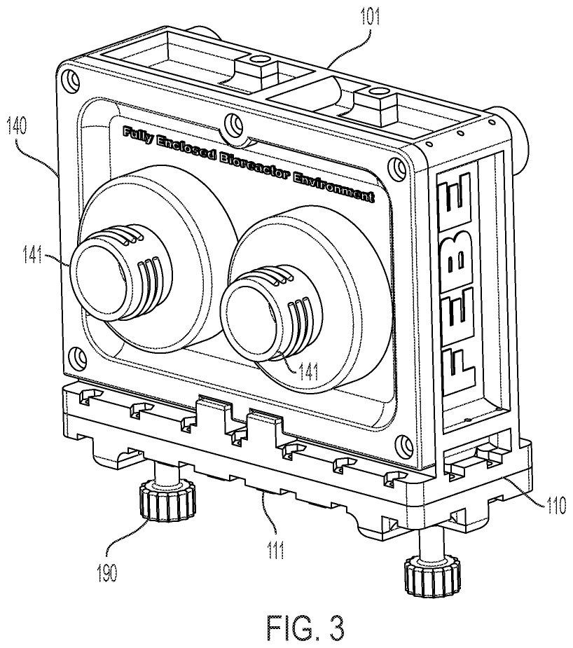

FIG. 3 schematically illustrates one embodiment of a bioreactor 100 comprising a bioreactor chamber 101, membrane wall 110 (portion of a perimeter of membrane wall) a lid structure 140, clamp screws 190, and a membrane mount structure 111.

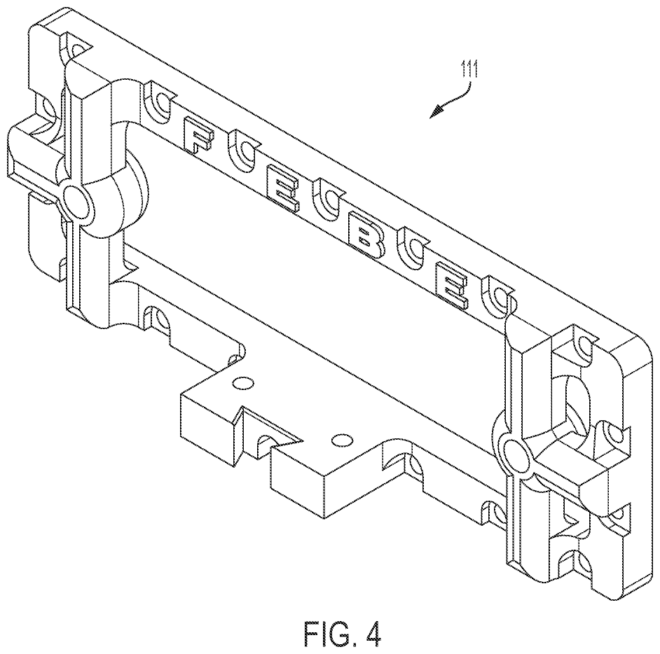

FIG. 4 schematically illustrates one embodiment of a membrane mount structure 111, wherein the membrane mount structure 111 may be an outline of the membrane wall 110 (not shown) that is applied to the outside of the membrane wall 110 (not shown in instant illustration) and pinches the membrane wall 110 (not shown in instant illustration) between the membrane mount structure 111 and the bioreactor chamber 101 (not shown in instant illustration), creating a sealed environment for the interior of the bioreactor 100 (not shown in instant illustration).

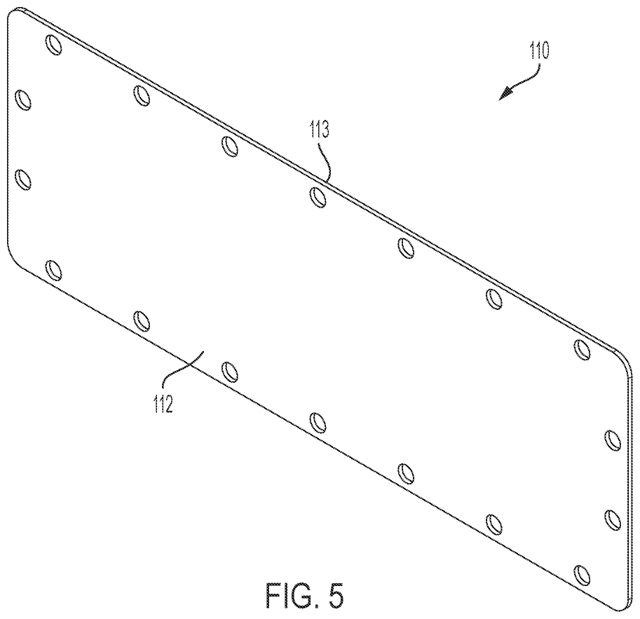

FIG. 5 schematically illustrates an embodiment of a membrane wall 110, having two surfaces, one inner surface 113 (not visible in instant illustration) and one outer surface 112.

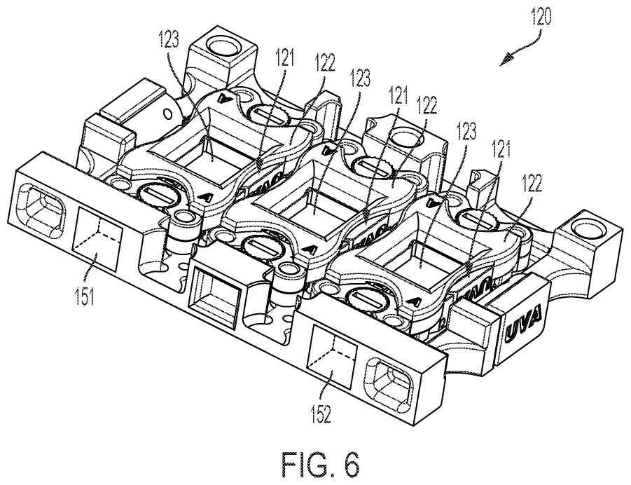

FIG. 6 schematically illustrates an embodiment of a scaffold structure 120 with cell seeding scaffolds 121 and associated inner coupling mechanisms 151, 152 (shown in dashed lines) (e.g., scaffold structure couplings). The scaffold structure 120 is able to stretch linearly to allow the cell seeding scaffolds 121 to stretch the cell material 123 and allow for optimal growth, while the cell seeding scaffolds 121 are configured so as to be able to be removed from the scaffold structure 120 to facilitate the seeding process and allow seeding on either side of the cell seeding scaffold 121.

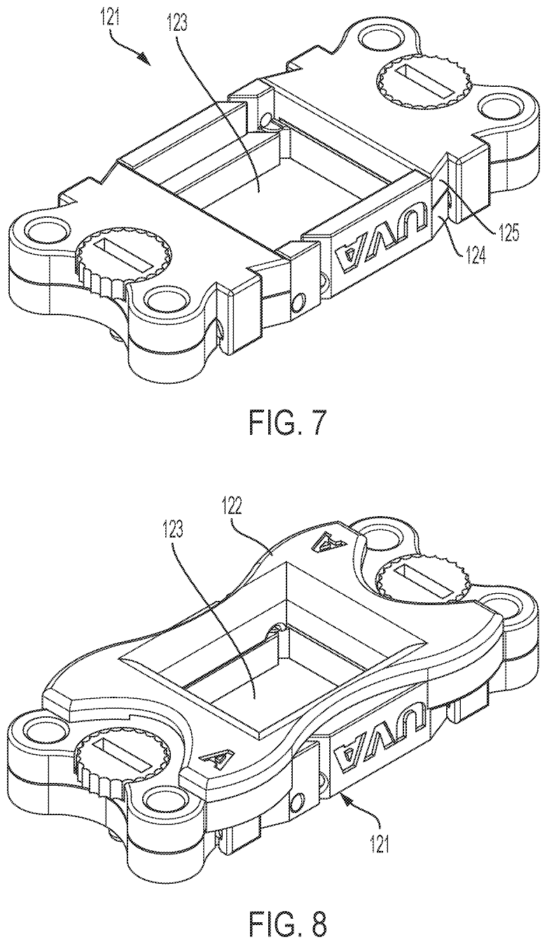

FIG. 7 shows a more detailed view of the cell seeding scaffold 121, having an open area (configured to hold or contain cell material 123) to allow for cell seeding and being made up of a top bracket 124 and bottom bracket 125 that are configured so as to be able to move away from one another linearly to facilitate the cell growth movement.

FIG. 8 schematically illustrates an embodiment of a cell seeding scaffold 121, to hold or contain the cell material 123, with a cell seeding brace 122. The cell seeding brace 122 is used to hold the cell seeding scaffold 121 stable while being transported or installed.

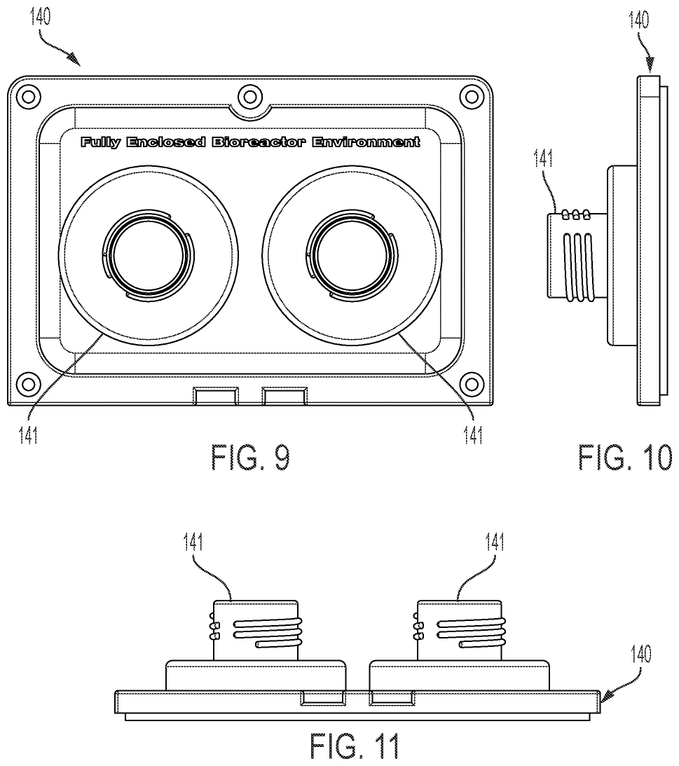

FIGS. 9-11 schematically illustrate an embodiment of a lid structure 140 which contains lid ports 141 in a top view, side view, and front view, respectively. These ports 141 allow for screw-on filters or the like to be attached, which allow for purified air to enter the bioreactor chamber 101 (not shown in instant Figure) without risking contamination of the cells of the cell material 123 (not shown in instant Figure). These lid ports 141 could also be used to allow for a nutrient fluid to flow into the bioreactor chamber 101 (not shown in instant Figure), so as to function such as a valve or the like, to aid in the cell growth process. These lid ports 141 could also be used to allow for nutrient exchange or the flow of oxygen, filtered oxygen, or other gases (or materials). Alternatively, or in combination thereof, the ports or other valves may be displaced or disposed on other locations of bioreactor chamber (not shown in instant Figure). In an embodiment the ports or valves may be located on any wall of the bioreactor chamber other than the lid. In an embodiment the ports or valves may be located on membrane wall.

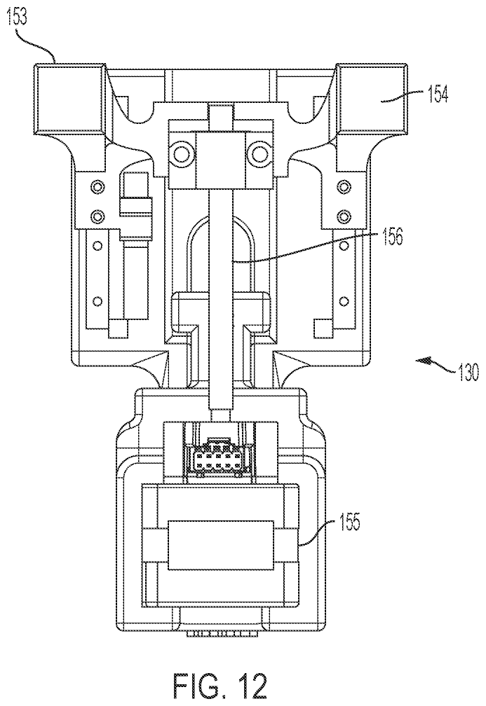

FIG. 12 schematically illustrates a top view of an embodiment of the linear transfer means comprising outer coupling mechanisms 153, 154 (e.g., linear actuator couplings). Also shown is the linear actuator 130 comprising a lead screw 156 and a motor 155. This Figure illustrates how the linear actuator 130 may be configured so as to be able to linearly advance the outer coupling mechanisms 153, 154. For example, the lead screw 156 in communication with the outer coupling mechanisms 153, 154 moves (any one or plurality of times) the outer coupling mechanisms 153, 154 forward and backward (e.g., inward and outward) in response to the motion of the lead screw 156 while keeping the coupling mechanism 159 at specified or designated positions to preserve the integrity of the membrane wall 110 (not shown in instant Figure).

FIGS. 13-28 schematically illustrate the steps of operation of an embodiment of a bioreactor 100. FIGS. 13, 15, 17, 19, 21, 23, 25, and 27 schematically illustrate a side view of the bioreactor 100 with the section view locations for FIGS. 14, 16, 18,20, 22, 24, 26, and 28, respectively, specified. Collectively, FIGS. 13-28 schematically illustrate the bioreactor 100 comprising, at least in part, the bioreactor 100, bioreactor chamber 101, membrane wall 110 (e.g., as illustrated depicting an edge perimeter of the membrane wall), membrane mount structure 111, scaffold structure 120, cell seeding scaffold 121, linear actuator 130, inner coupling mechanism 151, 152, outer coupling mechanism 153, 154, motor 155, lead screw 156, and clamps or screws 190.

FIG. 14 schematically illustrates a top section view (as specified in cross-section of FIG. 13) of the bioreactor 100 in the initial state after the scaffold structure 120 is installed, and neither the screws or clamps 190 nor the coupling mechanism is engaged (meaning the outer coupling mechanisms 153, 154 (e.g., linear actuator couplings) are not engaged with the inner coupling mechanisms 151, 152 (e.g., scaffold structure couplings)).

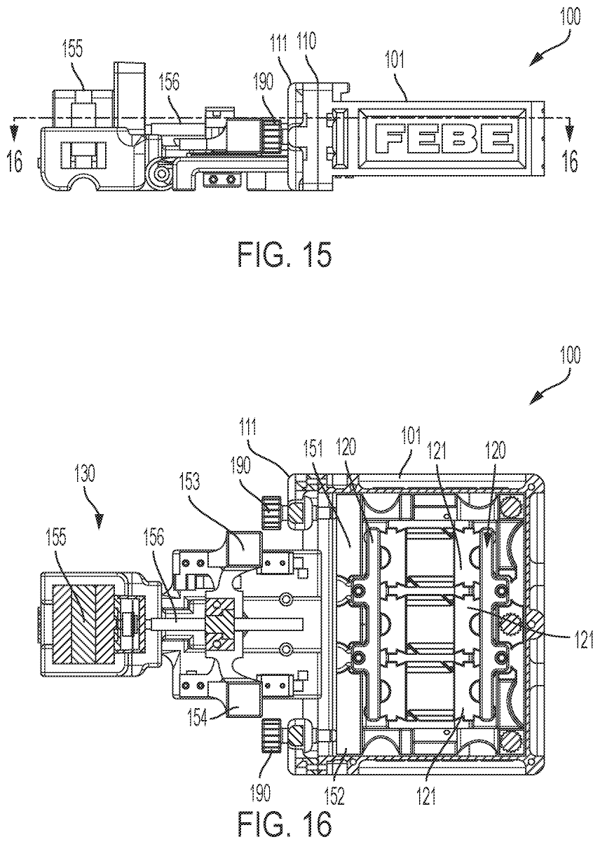

FIG. 16 schematically illustrates a top section view (as specified in cross-section of FIG. 15) of the bioreactor 100 in a state following that shown in FIG. 14, where the clamps or screws 190 are engaged in a way which prevents the scaffold structure 120 from moving, and the coupling mechanism is not engaged (meaning the outer coupling mechanisms 153, 154 (e.g., linear actuator couplings) are not engaged with the inner coupling mechanisms 151, 152 (e.g., scaffold structure couplings) on either side of the membrane wall 110).

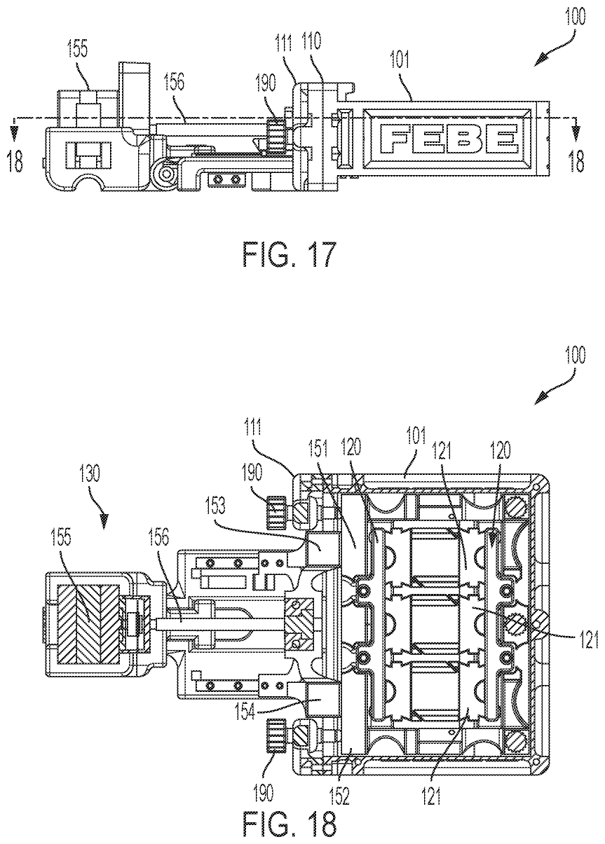

FIG. 18 schematically illustrates a top section view (as specified in cross-section of FIG. 17) of the bioreactor 100 in a state following that shown in FIG. 16, where the clamps or screws 190 remain engaged and the coupling mechanism is engaged (meaning the outer coupling mechanisms 153, 154 are engaged with the inner coupling mechanisms 151, 152 on either side of the membrane wall 110).

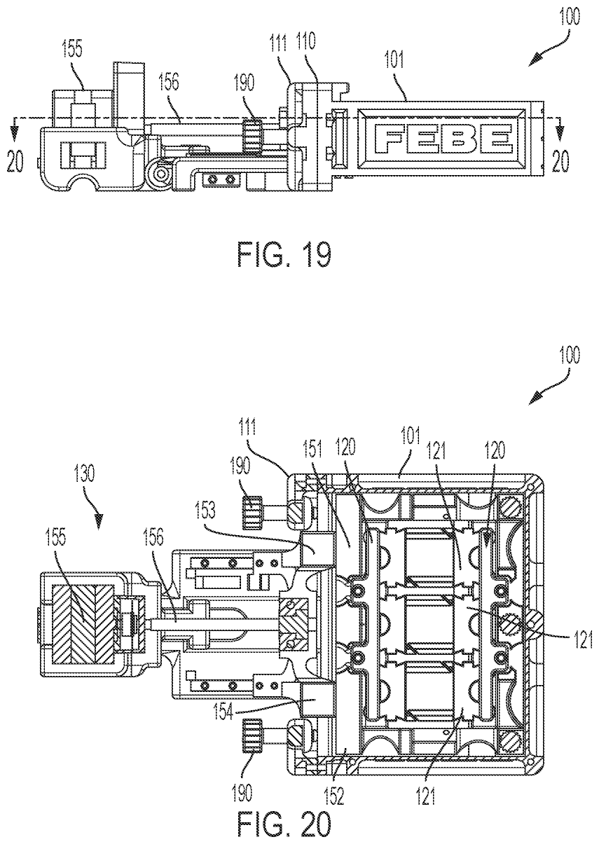

FIG. 20 schematically illustrates a top section view (as specified in cross-section of FIG. 19) of the bioreactor 100 in a state following that shown in FIG. 18, where the clamps or screws 190 are disengaged and the coupling mechanism remains engaged (meaning the outer coupling mechanisms 153, 154 are engaged with the inner coupling mechanisms 151, 152 on either side of the membrane wall 110).

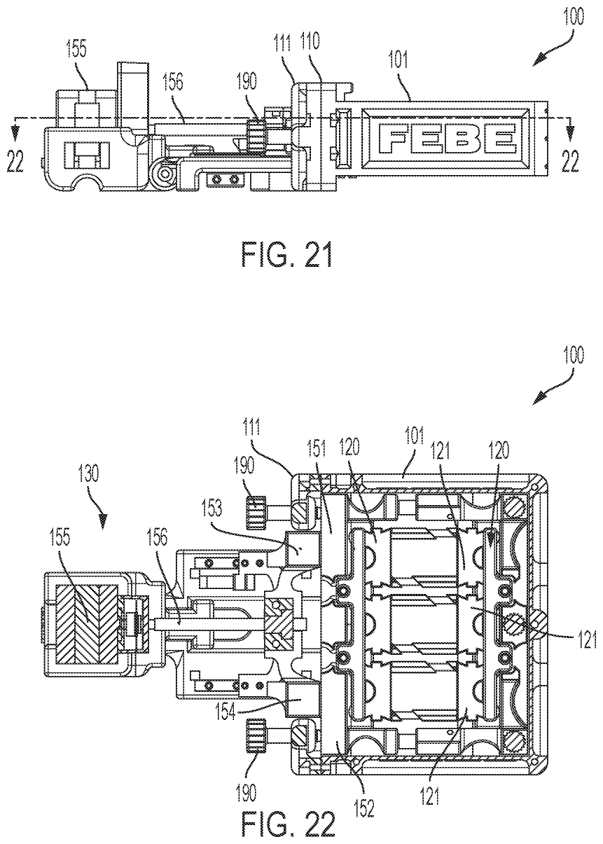

FIG. 22 schematically illustrates a top section view (as specified in cross-section of FIG. 21) of the bioreactor 100 in a state following that shown in FIG. 20, where the clamps or screws 190 remain disengaged, the coupling mechanism remains engaged (meaning the outer coupling mechanisms 153, 154 are engaged with the inner coupling mechanisms 151, 152 on either side of the membrane wall 110), and the linear actuator 130 (e.g., a lead screw 156) is retracted (in the leftward direction as oriented by the illustration) causing one side of the scaffold structure 120 to move (in the leftward direction as oriented by the illustration) and causing elongation of the cell seeding scaffold 121.

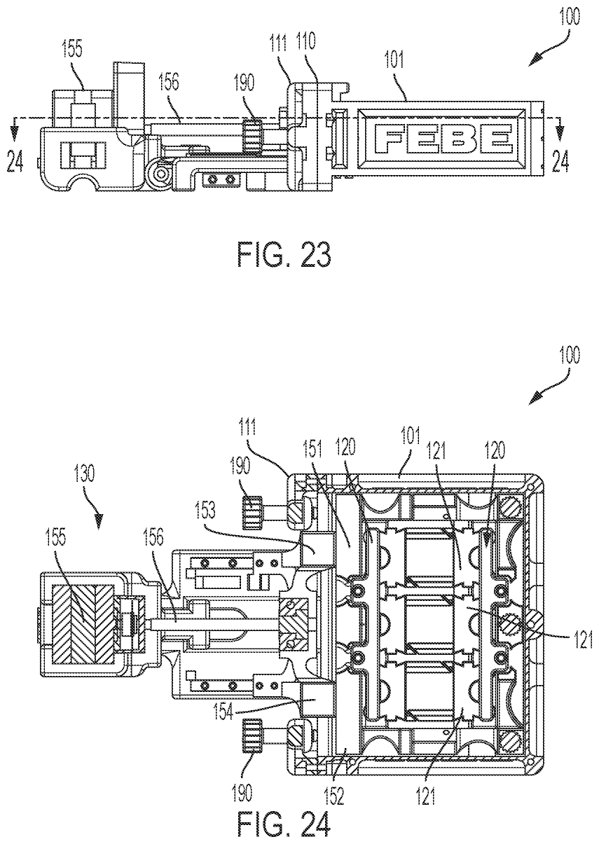

FIG. 24 schematically illustrates a top section view (as specified in cross-section of FIG. 23) of the bioreactor 100 in a state following that shown in FIG. 22, where the linear actuator 130 is returned to the starting or previous position in a non-elongated state (such as previously shown in FIG. 20) and while the clamps or screws 190 remain in a disengaged state and the coupling mechanism remains in an engaged state (meaning the outer coupling mechanisms 153, 154 remain engaged with the inner coupling mechanisms 151, 152 on either side of the membrane wall 110).

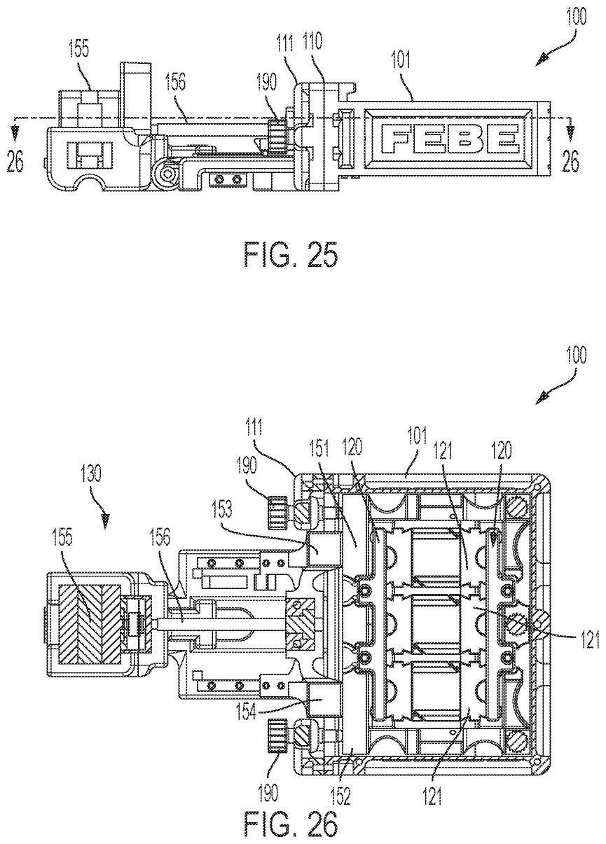

FIG. 26 schematically illustrates a top section view (as specified in cross-section of FIG. 25) of the bioreactor 100 in a state following that shown in FIG. 24, where the clamps or screws 190 are engaged in a way which prevents the scaffold structure 120 from moving, and the coupling mechanism remains engaged (meaning the outer coupling mechanisms 153, 154 remain engaged with the inner coupling mechanisms 151, 152 on either side of the membrane wall 110).

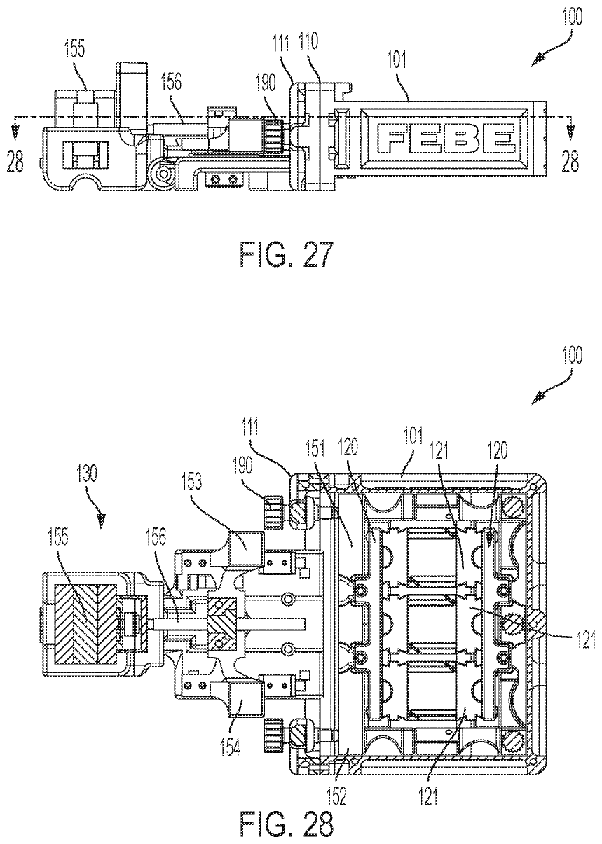

FIG. 28 schematically illustrates a top section view (as illustrated in cross-section of FIG. 27) of the bioreactor 100 in a state following that shown in FIG. 26, where the clamps or screws 190 remain engaged, and the coupling mechanism is disengaged (meaning the outer coupling mechanisms 153, 154 are disengaged from the inner coupling mechanisms 151, 152 on either side of the membrane wall 110).

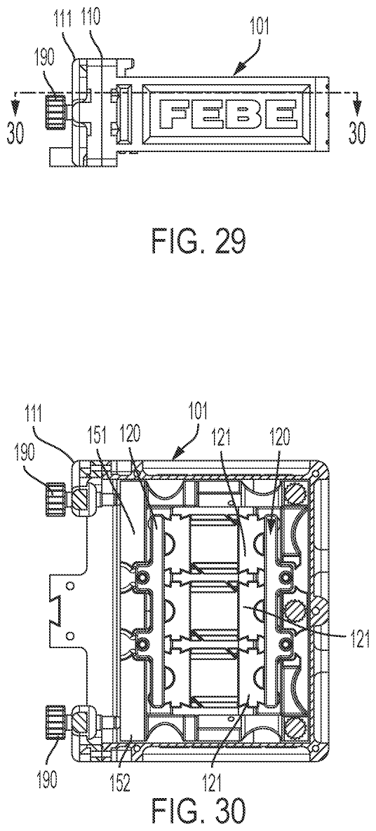

FIGS. 29 and 30 schematically illustrate an embodiment of a bioreactor chamber 101, side view and top view, respectively, wherein the bioreactor chamber 101 can be detached from the remainder of the bioreactor (not shown in instant Figure). This would allow the sealed bioreactor chamber 101 to be shipped, transported, or transferred separately from the remainder of the bioreactor while still maintaining a hermetic or aseptic seal. Alternatively, this would allow the sealed bioreactor chamber 101 to be viewed or accessed separately from the remainder of the bioreactor while still maintaining a hermetic or aseptic seal. FIG. 30 schematically illustrates the clamps or screws 190 in the position where the scaffold structure 120 is secured and prevented from moving.

FIGS. 31-36 schematically illustrate whereby the linear transfer means may be configured as a variety of, non-limiting, embodiments of the coupling mechanism 159.

FIG. 31 schematically illustrates an embodiment of a coupling mechanism 159 having the outer coupling mechanism 154 and inner coupling mechanism 152 wherein said outer coupling mechanism 154 and inner coupling mechanism 152 comprise an adhesive material 161 (whereby the inner coupling mechanism 152 and the outer coupling mechanism 154 are coupled together across the membrane 110 (spanning the membrane, with the membrane there between) without causing a breach in the membrane 110).

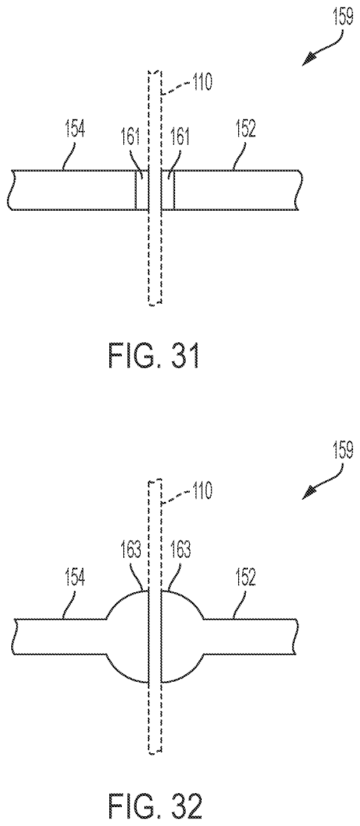

FIG. 32 schematically illustrates an embodiment of a coupling mechanism 159 having the outer coupling mechanism 154 and inner coupling mechanism 152 wherein said outer coupling mechanism 154 and inner coupling mechanism 152 comprises one or more suction cups 163 or other type of negative pressure connector (whereby the inner coupling mechanism 152 and the outer coupling mechanism 154 are coupled together across the membrane 110 (spanning the membrane, with the membrane there between) without causing a breach in the membrane 110).

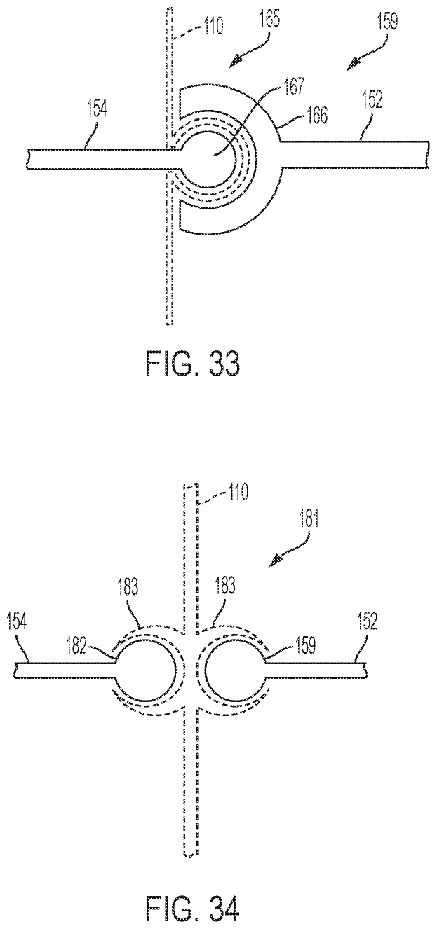

FIG. 33 schematically illustrates an embodiment of a coupling mechanism 159 having the outer coupling mechanism 154 and inner coupling mechanism 152 wherein said outer coupling mechanism 154 and inner coupling mechanism 152 comprises a buckle 165 that may include a frame 166 and prong 167 or other mechanical connector with a male and female component which attaches the inner and outer coupling mechanisms 152, 154 together across the membrane 110 (spanning the membrane, with the membrane there between) without causing a breach in the membrane 110.

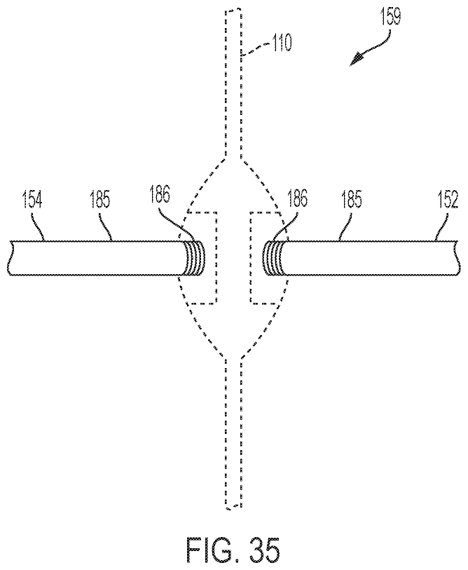

FIG. 34 schematically illustrates an embodiment of a coupling mechanism 159 having the outer coupling mechanism 154 and inner coupling mechanism 152 wherein said outer coupling mechanism 154 and inner coupling mechanism 152 comprises having one or more ball and socket joints 181 that may include a ball 182 and socket 183 that couples the outer coupling mechanism 154 and inner coupling mechanism 152 together across the membrane 110 (spanning the membrane, with the membrane there between), without causing a breach in the membrane 110, and wherein either the ball or socket side of the connector is attached or molded into the membrane.

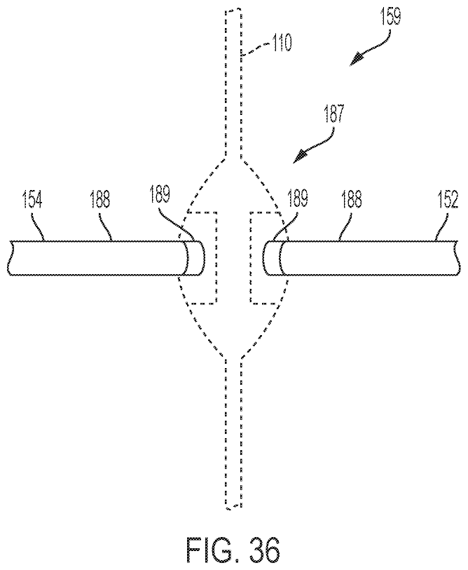

FIG. 35 schematically illustrates an embodiment of a coupling mechanism 159 having the outer coupling mechanism 154 and inner coupling mechanism 152 wherein said outer coupling mechanism 154 and inner coupling mechanism 152 comprise one or more threaded rod, screw, or bolt connectors 185 coupled by attachment to a threaded receiving portion or threaded socket 186 attached or molded into the membrane 110 (whereby the inner coupling mechanism 152 and the outer coupling mechanism 154 are coupled together across the membrane 110 (spanning the membrane, with the membrane there between) without causing a breach in the membrane 110).

FIG. 36 schematically illustrates an embodiment of a coupling mechanism 159 having the outer coupling mechanism 154 and inner coupling mechanism 152 wherein said outer coupling mechanism 154 and inner coupling mechanism 152 comprise one or more male-female 187 connectors that may include a male connector 188 and female connector 189 where the female side of the connector is attached or molded into the membrane 110, and the corresponding male side of the connector is located on the outer and/or inner coupling mechanism (whereby the inner coupling mechanism 152 and the outer coupling mechanism 154 are coupled together across the membrane 110 (spanning the membrane, with the membrane there between) without causing a breach in the membrane 110). In an alternative embodiment, the male side of the connector may be attached or molded into the membrane 110, and the corresponding female side of the connector is located on the outer and/or inner coupling mechanism.

### EXAMPLES

Practice of an aspect of an embodiment (or embodiments) of the invention will be still more fully understood from the following examples, which are presented herein for illustration only and should not be construed as limiting the invention in any way.

Example 1. A bioreactor comprising: a bioreactor chamber; a membrane wall disposed on said bioreactor chamber, wherein said bioreactor chamber and said membrane wall are configured to maintain sterility or sanitation within said bioreactor chamber while said bioreactor chamber is closed; a scaffold structure disposed inside said bioreactor chamber; a linear actuator disposed outside said bioreactor chamber; a linear transfer means for transferring linear motion between said linear actuator and said scaffold structure without breaching said membrane wall; and a control system in communication with said linear actuator configured to control the movement of said linear actuator.

Example 2. The bioreactor of example 1, wherein said bioreactor chamber and said membrane wall are configured to separate from said linear actuator, said linear transfer means, and said control system while maintaining the sterility or sanitation.

Example 3. The bioreactor of example 2, wherein said separated bioreactor chamber is configured for shipping, transporting, or transferring.

Example 4. The bioreactor of example 1 (as well as subject matter of one or more of any combination of examples 2-3, in whole or in part), wherein said membrane wall has one or more surfaces which form an outer boundary of said bioreactor chamber.

Example 5. The bioreactor of example 1 (as well as subject matter of one or more of any combination of examples 2-4, in whole or in part), wherein said membrane wall comprises at least one or more of the following materials: silicone, latex, or polymer.

Example 6. The bioreactor of example 1 (as well as subject matter of one or more of any combination of examples 2-6, in whole or in part), wherein said scaffold structure is configured to be able to move along a one directional axis.

Example 7. The bioreactor of example 1 (as well as subject matter of one or more of any combination of examples 2-6, in whole or in part), wherein said scaffold structure comprises one or more cell seeding scaffolds.

Example 8. The bioreactor of example 1 (as well as subject matter of one or more of any combination of examples 2-7, in whole or in part), wherein said scaffold structure is double sided.

Example 9. The bioreactor of example 1 (as well as subject matter of one or more of any combination of examples 2-8, in whole or in part), wherein said scaffold structure is configured to be removable from said bioreactor chamber when said bioreactor chamber is in an open position.

Example 10. The bioreactor of example 1 (as well as subject matter of one or more of any combination of examples 2-9, in whole or in part), wherein said bioreactor chamber is configured to allow repeated insertion and removal of said scaffold structure when said bioreactor chamber is in an open position.

Example 11. The bioreactor of example 1 (as well as subject matter of one or more of any combination of examples 2-10, in whole or in part), wherein said linear actuator is a stepper motor or servomotor.

Example 12. The bioreactor of example 1 (as well as subject matter of one or more of any combination of examples 2-11, in whole or in part), wherein said linear actuator is a rack and pinion.

Example 13. The bioreactor of example 1 (as well as subject matter of one or more of any combination of examples 2-12, in whole or in part), wherein said linear actuator is a piston.

Example 14. The bioreactor of example 13, wherein said piston is a pneumatic, magnetic or hydraulic type piston.

Example 15. The bioreactor of example 1 (as well as subject matter of one or more of any combination of examples 2-14, in whole or in part), wherein said linear actuator is a crank and slider mechanism.

Example 16. The bioreactor of example 1 (as well as subject matter of one or more of any combination of examples 2-15, in whole or in part), wherein said linear actuator is a solenoid.

Example 17. The bioreactor of example 1 (as well as subject matter of one or more of any combination of examples 2-16, in whole or in part), wherein said linear actuator is a leadscrew mechanism.

Example 18. The bioreactor of example 1 (as well as subject matter of one or more of any combination of examples 2-17, in whole or in part), wherein said linear actuator is configured to be activated cyclically or for specified time periods or durations.

Example 19. The bioreactor of example 1 (as well as subject matter of one or more of any combination of examples 2-18, in whole or in part), wherein said linear actuator is configured to be detachable from said bioreactor chamber while maintaining the sterility or sanitation.

Example 20. The bioreactor of example 1 (as well as subject matter of one or more of any combination of examples 2-19, in whole or in part), wherein said linear transfer means is comprised of a coupling mechanism, wherein said coupling mechanism is configured to couple said linear actuator to said scaffold structure, in response to said linear motion, on opposing sides of said membrane wall without breaching said membrane wall. The coupling mechanism comprises a linear actuator coupling that is disposed on said linear actuator; and wherein said coupling mechanism comprises a scaffold structure coupling that is disposed on said scaffold structure; wherein: either said linear actuator coupling or scaffold structure coupling is an adhesive material; or both said linear actuator coupling or scaffold structure coupling is an adhesive material.

Example 21. The bioreactor of example 1 (as well as subject matter of one or more of any combination of examples 2-20, in whole or in part), wherein said linear transfer means is comprised of a coupling mechanism, wherein said coupling mechanism is configured to couple said linear actuator to said scaffold structure, in response to said linear motion, on opposing sides of said membrane wall without breaching said membrane wall.

Example 22. The bioreactor of example 21, wherein said coupling mechanism comprises: at least one magnet disposed on said linear actuator to define a linear actuator magnet; and at least one magnet disposed on said scaffold structure to define a scaffold structure magnet, wherein said at least one linear actuator magnet and said at least one scaffold structure magnet are configured to join with one another, in response to said linear motion, so as to accomplish said coupling on opposing sides of said membrane wall without breaching said membrane wall.

Example 23. The bioreactor of example 22, wherein said at least one linear actuator magnet and said at least scaffold structure magnet are: permanent type magnets; electromagnet type magnets; or combination of both permanent magnet and electromagnet type magnets.

Example 24. The bioreactor of example 21 (as well as subject matter of one or more of any combination of examples 2-23, in whole or in part), wherein said coupling mechanism comprises: at least one magnet disposed on said linear actuator to define a linear actuator magnet; and at least one ferromagnetic material device disposed on said scaffold structure to define a scaffold structure ferromagnetic material device, wherein said at least one linear actuator magnet and said at least one scaffold structure ferromagnetic material device are configured to join with one another, in response to said linear motion, so as to accomplish said coupling on opposing sides of said membrane wall without breaching said membrane wall.

Example 25. The bioreactor of example 21 (as well as subject matter of one or more of any combination of examples 2-24, in whole or in part), wherein said coupling mechanism comprises: at least one ferromagnetic material device disposed on said linear actuator to define a linear actuator magnet; and at least one magnet disposed on said scaffold structure to define a scaffold structure magnet, wherein said at least one linear actuator ferromagnetic material device and said at least one scaffold structure magnet are configured to join with one another, in response to said linear motion, so as to accomplish said coupling on opposing sides of said membrane wall without breaching said membrane wall.

Example 26. The bioreactor of example 1 (as well as subject matter of one or more of any combination of examples 2-25, in whole or in part), wherein said linear transfer means is comprised of a coupling mechanism, wherein said coupling mechanism is configured to couple said linear actuator to said scaffold structure, in response to said linear motion, on opposing sides of said membrane wall without breaching said membrane wall; wherein said coupling mechanism comprises a linear actuator coupling that is disposed on said linear actuator. The coupling mechanism comprises a scaffold structure coupling that is disposed on said scaffold structure; wherein: either said linear actuator coupling or scaffold structure coupling is a suction cup; or both said linear actuator coupling or scaffold structure coupling is a suction cup.

Example 27. The bioreactor of example 21 (as well as subject matter of one or more of any combination of examples 2-26, in whole or in part), wherein said coupling mechanism comprises: a buckle device, wherein said buckle device includes a first buckle and a second buckle, wherein said first buckle is disposed on the linear actuator and said second buckle is disposed on the scaffold structure, wherein said first buckle and said second buckle are configured to join with one another, in response to said linear motion, so as to accomplish said coupling on opposing sides of said membrane wall without breaching said membrane wall.

Example 28. The bioreactor of example 1 (as well as subject matter of one or more of any combination of examples 2-27, in whole or in part), wherein said linear transfer means is comprised of a coupling mechanism, wherein said coupling mechanism is configured to couple said linear actuator to said scaffold structure, in response to said linear motion, on opposing sides of said membrane wall without breaching said membrane wall; wherein said coupling mechanism comprises a linear actuator coupling that is disposed on said linear actuator and comprises an outer surface ball; and wherein said coupling mechanism comprises a scaffold structure coupling that is disposed on said scaffold structure and comprises an inner surface ball; wherein said membrane wall has an outer surface and an inner surface, wherein said outer surface comprises an outer socket configured to receive said outer surface ball and said inner surface comprises an inner socket configured to receive said inner surface ball.

Example 29. The bioreactor of example 1 (as well as subject matter of one or more of any combination of examples 2-28, in whole or in part), wherein said linear transfer means is comprised of a coupling mechanism, wherein said coupling mechanism is configured to couple said linear actuator to said scaffold structure, in response to said linear motion, on opposing sides of said membrane wall without breaching said membrane wall; wherein said coupling mechanism comprises a linear actuator coupling that is disposed on said linear actuator and comprises an outer surface screw; and wherein said coupling mechanism comprises a scaffold structure coupling that is disposed on said scaffold structure and comprises an inner surface screw; wherein said membrane wall has an outer surface and an inner surface, wherein said outer surface comprises an outer threaded socket configured to receive said outer surface screw and said inner surface comprises an inner threaded socket configured to receive said inner surface screw.

Example 30. The bioreactor of example 1 (as well as subject matter of one or more of any combination of examples 2-29, in whole or in part), wherein said linear transfer means is comprised of a coupling mechanism, wherein said coupling mechanism is configured to couple said linear actuator to said scaffold structure, in response to said linear motion, on opposing sides of said membrane wall without breaching said membrane wall; wherein said coupling mechanism comprises a linear actuator coupling that is disposed on said linear actuator and comprises an outer surface male connector; and wherein said coupling mechanism comprises a scaffold structure coupling that is disposed on said scaffold structure and comprises an inner surface male connector; wherein said membrane wall has an outer surface and an inner surface, wherein said outer surface comprises an outer female socket configured to receive said outer surface male connector and said inner surface comprises an inner female socket configured to receive said inner surface male connector.

Example 31. The bioreactor of example 21 (as well as subject matter of one or more of any combination of examples 2-30, in whole or in part), wherein said coupling mechanism is permanently or temporarily attached to said membrane.

Example 32. The bioreactor of example 21 (as well as subject matter of one or more of any combination of examples 2-31, in whole or in part), wherein said coupling mechanism is permanently or temporarily coupling said linear actuator to said scaffold structure.

Example 33. The bioreactor of example 1 (as well as subject matter of one or more of any combination of examples 2-32, in whole or in part), wherein said bioreactor chamber is configured to be hermetically sealed.

Example 34. The bioreactor of example 1 (as well as subject matter of one or more of any combination of examples 2-33, in whole or in part), wherein said bioreactor chamber is configured to be aseptically sealed.

Example 35. The bioreactor of example 1 (as well as subject matter of one or more of any combination of examples 2-34, in whole or in part), wherein the inner portion of said bioreactor chamber does not contain exposed metal surfaces.

Example 36. The bioreactor of example 1 (as well as subject matter of one or more of any combination of examples 2-35, in whole or in part), wherein the control system and linear actuator are disposed outside of the bioreactor chamber, and do not permeate the bioreactor chamber.

Example 37. The bioreactor of example 1 (as well as subject matter of one or more of any combination of examples 2-36, in whole or in part), further comprising one or more ports disposed on said bioreactor chamber.

Example 38. The bioreactor of example 1 (as well as subject matter of one or more of any combination of examples 2-37, in whole or in part), wherein said bioreactor chamber is configured to permit gas and/or nutrient exchange.

Example 39. The bioreactor of example 1 (as well as subject matter of one or more of any combination of examples 2-38, in whole or in part), further comprising a removable lid assembly.

Example 40. The bioreactor of example 39, wherein said removable lid assembly has one or more ports for the flow of gases and/or nutrients.

Example 41. The bioreactor of example 39 (as well as subject matter in whole or in part of example 40), wherein said removable lid assembly is configured to permit gas and/or nutrient exchange.

Example 42. The bioreactor of example 1 (as well as subject matter of one or more of any combination of examples 2-41, in whole or in part), wherein said membrane wall has sufficient flexibility whereby said membrane wall can be displaced resultant to said linear motion in a linear direction for a distance of one the following:
a range of about 1 mm to about 10 mm;
a range of about 1 mm to about 5 mm;
a range of about 1 mm to about 6 mm;
a range of about 2 mm to about 4 mm; or
about 3 mm.

Example 43. The bioreactor of example 1 (as well as subject matter of one or more of any combination of examples 2-42, in whole or in part), wherein said membrane wall has sufficient flexibility whereby said membrane wall can be displaced resultant to said linear motion in a linear direction for a distance of one the following:
a range of about 1 mm to about 10 cm;
a range of about 10 cm to about 1 m; or
a range of about 1 m to about 3 m.

Example 44. The bioreactor of example 21 (as well as subject matter of one or more of any combination of examples 2-43, in whole or in part), wherein said membrane wall has sufficient flexibility in the linear direction so as to permit said linear actuator and said scaffold structure to travel with respect to one another, in response to said linear motion, causing said membrane wall to flex in an ample manner so as to allow said linear actuator and said scaffold structure to couple with one another.

Example 45. The bioreactor of example 1 (as well as subject matter of one or more of any combination of examples 2-44, in whole or in part), wherein said membrane wall has sufficient elasticity in the linear direction so as to permit said linear actuator and said scaffold structure to travel with respect to one another, in response to said linear motion, causing said membrane wall to stretch in an ample manner so as to allow said linear actuator and said scaffold structure to couple with one another.

Example 46. The bioreactor of example 1 (as well as subject matter of one or more of any combination of examples 2-45, in whole or in part), wherein said membrane wall has sufficient deformability in the linear direction so as to permit said linear actuator and said scaffold structure to travel with respect to one another, in response to said linear motion, causing said membrane wall to deform in an ample manner so as to allow said linear actuator and said scaffold structure to couple with one another.

Example 47. The bioreactor of example 21 (as well as subject matter of one or more of any combination of examples 2-46, in whole or in part), wherein said membrane wall is configured to allow movement in the linear direction so as to permit said linear actuator and said scaffold structure to travel with respect to one another, in response to said linear motion, causing said membrane wall to move in an ample manner so as to allow said linear actuator and said scaffold structure to couple with one another.

Example 48. The bioreactor of example 1 (as well as subject matter of one or more of any combination of examples 2-47, in whole or in part), further comprising a securement means for securing said scaffold structure in place.

Example 49. The bioreactor of example 48, wherein said securement means is a clamp or screw adjustably mounted to said chamber wherein said clamp or screw is configured to make contact with said membrane to impart a force on said membrane to be transferred to said scaffold structure for maintaining a desired position of said scaffold structure.

Example 50. The bioreactor of example 1 (as well as subject matter of one or more of any combination of examples 2-49, in whole or in part), wherein said bioreactor chamber includes any one of the following structures: housing, enclosure, box, container, casing, tank, compartment, cavity, pipe, or trunk.

Example 51. A bioreactor device, said device comprising: a bioreactor chamber and a membrane wall disposed on said bioreactor chamber, wherein said bioreactor chamber is configured to hold a scaffold structure or other component; and said membrane wall is configured to allow transfer of linear motion to said scaffold structure or other component without breaching said membrane wall.

Example 52. The bioreactor device of example 51, wherein said bioreactor chamber said membrane wall are configured to maintain sterility or sanitation within said bioreactor chamber while said bioreactor chamber is closed.

Example 53. The device of example 51 (as well as subject matter in whole or in part of example 52), wherein said linear motion is a type of motion that can be generated by a linear actuator disposed outside said bioreactor chamber.

Example 54. The device of example 51 (as well as subject matter of one or more of any combination of examples 52-53, in whole or in part), wherein said device is provided as part of a kit, wherein said kit includes a linear actuator, wherein said linear actuator is configured to provide said linear motion.

Example 55. A system configured to receive said bioreactor device of example 51 (as well as subject matter of one or more of any combination of examples 52-54, in whole or in part), said system comprising: a linear actuator disposed outside said bioreactor chamber, wherein said linear actuator is configured to provide said linear motion; and a linear transfer means for transferring linear motion between said linear actuator and said scaffold structure or said other component without breaching said membrane wall.

Example 56. The system of example 54 (as well as subject matter of one or more of any combination of examples 52-55, in whole or in part), further comprising: a control system in communication with said linear actuator configured to control the movement of said linear actuator.

Example 57. The system of example 56, wherein said linear transfer means is comprised of a coupling mechanism, wherein said coupling mechanism is configured to couple said linear actuator to said scaffold structure, in response to said linear motion, on opposing sides of said membrane wall without breaching said membrane wall.

Example 58. The system of example 55 (as well as subject matter of one or more of any combination of examples 52-57, in whole or in part), wherein said linear transfer means is comprised of a coupling mechanism, wherein said coupling mechanism is configured to couple said linear actuator to said scaffold structure, in response to said linear motion, on opposing sides of said membrane wall without breaching said membrane wall.

Example 59. A bioreactor comprising: a bioreactor chamber; a membrane wall disposed on said bioreactor chamber; a scaffold structure disposed inside said bioreactor chamber;
a linear actuator disposed outside said bioreactor chamber; a linear transfer means for transferring linear motion between said linear actuator and said scaffold structure without breaching said membrane wall; and a control system in communication with said linear actuator configured to control the movement of said linear actuator.

Example 60. The bioreactor of example 59, wherein said bioreactor chamber and said membrane wall are configured to separate from said linear actuator, said linear transfer means, and said control system.

Example 61. The bioreactor of example 60, wherein said bioreactor chamber and membrane wall are configured to maintain sterility or sanitation within said bioreactor chamber while said bioreactor is closed.

Example 62. The bioreactor of example 59 (as well as subject matter of one or more of any combination of examples 60-61, in whole or in part), wherein said bioreactor chamber and said membrane wall is configured to maintain sterility or sanitation within said bioreactor chamber while said bioreactor is closed.

Example 63. The bioreactor of example 59 (as well as subject matter of one or more of any combination of examples 60-62, in whole or in part), wherein said linear transfer means is comprised of a coupling mechanism, wherein said coupling mechanism is configured to couple said linear actuator to said scaffold structure, in response to said linear motion, on opposing sides of said membrane wall without breaching said membrane wall.

Example 64. A bioreactor comprising: a bioreactor chamber; a membrane wall disposed on said bioreactor chamber, wherein said bioreactor chamber and said membrane wall are configured to maintain sterility or sanitation within said bioreactor chamber while said bioreactor chamber is closed; a scaffold structure disposed inside said bioreactor chamber; an actuator disposed outside said bioreactor chamber; a transfer means for transferring motion between said actuator and said scaffold structure without breaching said membrane wall; and a control system in communication with said actuator configured to control the movement of said actuator.

Example 65. The bioreactor of example 64 further comprising any of the elements, components, systems, devices, materials, or their sub-components, provided in any one or more of examples 1-50 or 59, in whole or in part.

Example 66. A method of manufacturing any of the elements, components, systems, devices, materials, or their sub-components, provided in any one or more of examples 1-63, in whole or in part.

Example 67. A method of using any of the bioreactors, systems, elements, components, devices, materials, or their sub-components, provided in any one or more of examples 1-63, in whole or in part.

Example 68. A method of transporting or transferring any of the bioreactors, systems, elements, components, devices, materials, or their sub-components, provided in any one or more of examples 1-63, in whole or in part.

Example 69. A method of manufacturing cells and tissues using any of the bioreactors, systems, elements, components, devices, materials, or their sub-components, provided in any one or more of examples 1-63, in whole or in part.

Example 70. A method of stimulating cell growth and/or maturation using any of the bioreactors, systems, elements, components, devices, materials, or their sub-components, provided in any one or more of examples 1-63, in whole or in part.

Example 71. Cells and/or tissues manufactured using the methods provided in any one or more of examples 69-70, in whole or in part.

Example 72. Cells and/or tissues manufactured using any of the bioreactors, systems, elements, components, devices, materials, or their sub-components, provided in any one or more of examples 1-63, in whole or in part.

Example 73. A system configured to perform the method of any one or more of examples 69-70.

Example 74. The bioreactor device of example 51 further comprising any of the elements, components, systems, devices, materials, or their sub-components, provided in any one or more of examples 2-48, in whole or in part.

Example 75. The bioreactor device of example 55 further comprising any of the elements, components, systems, devices, materials, or their sub-components, provided in any one or more of examples 2-48, in whole or in part.

Example 76. An article of manufacture that is manufactured using the methods provided in any one or more of examples 69-70, in whole or in part.

Example 77. An article of manufacture that is manufactured using any of the bioreactors, systems, elements, components, devices, materials, or their sub-components, provided in any one or more of examples 1-63, in whole or in part.

### REFERENCES

The devices, systems, apparatuses, modules, compositions, articles of manufacture, materials, computer program products, non-transitory computer readable medium, and methods of various embodiments of the invention disclosed herein may utilize aspects (such as devices, apparatuses, modules, systems, compositions, articles of manufacture, materials, computer program products, non-transitory computer readable medium, and methods) disclosed in the following references, applications, publications and patents and which are hereby incorporated by reference herein in their entirety (and which are not admitted to be prior art with respect to the present invention by inclusion in this section).
1. U.S. Utility patent application Ser. No. 17/282,117 entitled “MODULAR BIOFABRICATION PLATFORM FOR DIVERSE TISSUE ENGINEERING APPLICATIONS AND RELATED METHOD THEREOF”, filed Apr. 1, 2021.
2. International Patent Application Serial No. PCT/US2019/054744 entitled “MODULAR BIOFABRICATION PLATFORM FOR DIVERSE TISSUE ENGINEERING APPLICATIONS AND RELATED METHOD THEREOF”, filed Oct. 4, 2019; Publication No. WO 2020/072933, Apr. 9, 2020.
3. U.S. Utility patent application Ser. No. 17/049,237 entitled “USE OF A HYALURONIC ACID-BASED HYDROGEL FOR TREATMENT OF VOLUMETRIC MUSCLE LOSS INJURY”, filed Oct. 20, 2020.
4. International Patent Application Serial No. PCT/US2019/028558 entitled “USE OF A HYALURONIC ACID-BASED HYDROGEL FOR TREATMENT OF VOLUMETRIC MUSCLE LOSS INJURY”, filed Apr. 22, 2019; Publication No. WO 2019/204818, Oct. 24, 2019.
5. U.S. Utility patent application Ser. No. 16/322,691 entitled “BIOREACTOR CONTROLLER DEVICE AND RELATED METHOD THEREOF”, filed Feb. 1, 2019.
6. International Patent Application Serial No. PCT/US2017/045299 entitled “BIOREACTOR CONTROLLER DEVICE AND RELATED METHOD THEREOF”, filed Aug. 3, 2017; Publication No. WO 2018/027033, Feb. 8, 2018.
7. U.S. Utility patent application Ser. No. 15/760,009 entitled “BIOREACTOR AND RESEEDING CHAMBER SYSTEM AND RELATED METHODS THEREOF”, filed Mar. 14, 2018; Publication No. US-2018-0265831-A1, Sep. 20, 2018.
8. International Patent Application Serial No. PCT/US2016/051948 entitled “BIOREACTOR AND RESEEDING CHAMBER SYSTEM AND RELATED METHODS THEREOF”, filed Sep. 15, 2016; Publication No. WO 2017/048961, Mar. 23, 2017.
9. U.S. Utility patent application Ser. No. 15/770,413, entitled “DEVICES, SYSTEMS AND METHODS FOR SAMPLE DETECTION”, filed Apr. 23, 2018; Publication No. US-2019-0054468-A1, Feb. 21, 2019.
10. International Patent Application Serial No. PCT/US2016/058263, entitled “DEVICES, SYSTEMS AND METHODS FOR SAMPLE DETECTION”, filed Oct. 21, 2016; Publication No. WO 2017/070571, Apr. 27, 2017.
11. U.S. Pat. No. 7,399,168 B1, Eberwein, “Air Driven Diaphragm Pump”, Jul. 15, 2008. Tapflo catalogue, “Air Operated Diaphragm Pumps”, 2013 (Rev. 1). https://www.tapflo.co.jp/images/Diaphragm_pumps_40_pages_catalogue_english.en.pdf
12. U.S. Pat. No. 7,695,967 B1, Russell, et al., “Method of Growing Stem Cells on a Membrane Containing Projections and Grooves”, Apr. 13, 2010.
13. U.S. Pat. No. 6,472,202 B1, Banes, “Loading Station Assembly and Method for Tissue Engineering”, Oct. 29, 2002.
14. U.S. Patent Application Publication No. US 2012/0100602 A1, Lu, et al., “Bioreactor System for Mechanical Stimulation of Biological Samples”, Apr. 26, 2012.
15. U.S. Patent Application Publication No. US 2011/0172683 A1, Yoo, et al., “Tissue Expander”, Jul. 14, 2011.
16. U.S. Patent Application Publication No. US 2018/0093015 A1, Murphy, et al., “Devices, Systems, and Methods for the Fabrication of Tissue”, Apr. 5, 2018.
17. U.S. Patent Application Publication No. US 2018/0265831 A1, Cao, et al., “Bioreactor and Reseeding Chamber System and Related Methods Thereof”, Sep. 20, 2018.
18. Korean Patent No. KR 10-1585328 B1, Kim, et al., “Hybrid Bio Print Apparatus for Manufacturing Scaffold Supporter and Method for Manufacturing Using the Same”, Jan. 14, 2016.

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes which come within the meaning and range of equivalency of the claims are therefore intended to be embraced herein.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particular interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub-ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

What is claimed is:

1. A bioreactor comprising:
a bioreactor chamber;
a membrane wall disposed on said bioreactor chamber, wherein said bioreactor chamber and said membrane wall are configured to maintain sterility or sanitation within said bioreactor chamber while said bioreactor chamber is closed;
a scaffold structure disposed inside said bioreactor chamber;
a linear actuator disposed outside said bioreactor chamber;
a linear transfer means for transferring linear motion between said linear actuator and said scaffold structure without breaching said membrane wall; and
a control system in communication with said linear actuator configured to control the movement of said linear actuator.

2. The bioreactor of claim 1, wherein said bioreactor chamber and said membrane wall are configured to separate from said linear actuator, said linear transfer means, and said control system while maintaining the sterility or sanitation.

3. The bioreactor of claim 2, wherein said separated bioreactor chamber is configured for shipping, transporting, or transferring.

4. The bioreactor of claim 1, wherein said membrane wall has one or more surfaces which form an outer boundary of said bioreactor chamber.

5. The bioreactor of claim 1, wherein said membrane wall comprises at least one or more of the following materials: silicone, latex, or polymer.

6. The bioreactor of claim 1, wherein said scaffold structure is configured to be able to move along a one directional axis.

7. The bioreactor of claim 1, wherein said scaffold structure comprises one or more cell seeding scaffolds.

8. The bioreactor of claim 1, wherein said scaffold structure is double sided.

9. The bioreactor of claim 1, wherein said scaffold structure is configured to be removable from said bioreactor chamber when said bioreactor chamber is in an open position.

10. The bioreactor of claim 1, wherein said bioreactor chamber is configured to allow repeated insertion and removal of said scaffold structure when said bioreactor chamber is in an open position.

11. The bioreactor of claim 1, wherein said linear actuator is a stepper motor or servomotor.

12. The bioreactor of claim 1, wherein said linear actuator is a rack and pinion.

13. The bioreactor of claim 1, wherein said linear actuator is a piston.

14. The bioreactor of claim 13, wherein said piston is a pneumatic, magnetic or hydraulic type piston.

15. The bioreactor of claim 1, wherein said linear actuator is a crank and slider mechanism.

16. The bioreactor of claim 1, wherein said linear actuator is a solenoid.

17. The bioreactor of claim 1, wherein said linear actuator is a leadscrew mechanism.

18. The bioreactor of claim 1, wherein said linear actuator is configured to be activated cyclically or for specified time periods or durations.

19. The bioreactor of claim 1, wherein said linear actuator is configured to be detachable from said bioreactor chamber while maintaining the sterility or sanitation.

20. The bioreactor of claim 1, wherein said linear transfer means is comprised of a coupling mechanism, wherein said coupling mechanism is configured to couple said linear actuator to said scaffold structure, in response to said linear motion, on opposing sides of said membrane wall without breaching said membrane wall;
wherein said coupling mechanism comprises a linear actuator coupling that is disposed on said linear actuator; and
wherein said coupling mechanism comprises a scaffold structure coupling that is disposed on said scaffold structure; wherein:
either said linear actuator coupling or scaffold structure coupling is an adhesive material; or
both said linear actuator coupling or scaffold structure coupling is an adhesive material.

21. The bioreactor of claim 1, wherein said linear transfer means is comprised of a coupling mechanism, wherein said coupling mechanism is configured to couple said linear actuator to said scaffold structure, in response to said linear motion, on opposing sides of said membrane wall without breaching said membrane wall.

22. The bioreactor of claim 21, wherein said coupling mechanism comprises:
at least one magnet disposed on said linear actuator to define a linear actuator magnet; and
at least one magnet disposed on said scaffold structure to define a scaffold structure magnet, wherein said at least one linear actuator magnet and said at least one scaffold structure magnet are configured to join with one another, in response to said linear motion, so as to accomplish said coupling on opposing sides of said membrane wall without breaching said membrane wall.

23. The bioreactor of claim 22, wherein said at least one linear actuator magnet and said at least scaffold structure magnet are:
permanent type magnets;
electromagnet type magnets; or
combination of both permanent magnet and electromagnet type magnets.

24. The bioreactor of claim 21, wherein said coupling mechanism comprises:
at least one magnet disposed on said linear actuator to define a linear actuator magnet; and
at least one ferromagnetic material device disposed on said scaffold structure to define a scaffold structure ferromagnetic material device, wherein said at least one linear actuator magnet and said at least one scaffold structure ferromagnetic material device are configured to join with one another, in response to said linear motion, so as to accomplish said coupling on opposing sides of said membrane wall without breaching said membrane wall.

25. The bioreactor of claim 21, wherein said coupling mechanism comprises:
at least one ferromagnetic material device disposed on said linear actuator to define a linear actuator magnet; and
at least one magnet disposed on said scaffold structure to define a scaffold structure magnet, wherein said at least one linear actuator ferromagnetic material device and said at least one scaffold structure magnet are configured to join with one another, in response to said linear motion, so as to accomplish said coupling on opposing sides of said membrane wall without breaching said membrane wall.

26. The bioreactor of claim 1, wherein said linear transfer means is comprised of a coupling mechanism, wherein said coupling mechanism is configured to couple said linear actuator to said scaffold structure, in response to said linear motion, on opposing sides of said membrane wall without breaching said membrane wall;
wherein said coupling mechanism comprises a linear actuator coupling that is disposed on said linear actuator; and
wherein said coupling mechanism comprises a scaffold structure coupling that is disposed on said scaffold structure; wherein:
either said linear actuator coupling or scaffold structure coupling is a suction cup; or
both said linear actuator coupling or scaffold structure coupling is a suction cup.

27. The bioreactor of claim 21, wherein said coupling mechanism comprises:
a buckle device, wherein said buckle device includes a first buckle and a second buckle, wherein said first buckle is disposed on the linear actuator and said second buckle is disposed on the scaffold structure, wherein said first buckle and said second buckle are configured to join with one another, in response to said linear motion, so as to accomplish said coupling on opposing sides of said membrane wall without breaching said membrane wall.

28. The bioreactor of claim 1, wherein said linear transfer means is comprised of a coupling mechanism, wherein said coupling mechanism is configured to couple said linear actuator to said scaffold structure, in response to said linear motion, on opposing sides of said membrane wall without breaching said membrane wall;
wherein said coupling mechanism comprises a linear actuator coupling that is disposed on said linear actuator and comprises an outer surface ball; and
wherein said coupling mechanism comprises a scaffold structure coupling that is disposed on said scaffold structure and comprises an inner surface ball;
wherein said membrane wall has an outer surface and an inner surface, wherein said outer surface comprises an outer socket configured to receive said outer surface ball and said inner surface comprises an inner socket configured to receive said inner surface ball.

29. The bioreactor of claim 1, wherein said linear transfer means is comprised of a coupling mechanism, wherein said coupling mechanism is configured to couple said linear actuator to said scaffold structure, in response to said linear motion, on opposing sides of said membrane wall without breaching said membrane wall;
wherein said coupling mechanism comprises a linear actuator coupling that is disposed on said linear actuator and comprises an outer surface screw; and
wherein said coupling mechanism comprises a scaffold structure coupling that is disposed on said scaffold structure and comprises an inner surface screw;
wherein said membrane wall has an outer surface and an inner surface, wherein said outer surface comprises an outer threaded socket configured to receive said outer surface screw and said inner surface comprises an inner threaded socket configured to receive said inner surface screw.

30. The bioreactor of claim 1, wherein said linear transfer means is comprised of a coupling mechanism, wherein said coupling mechanism is configured to couple said linear actuator to said scaffold structure, in response to said linear motion, on opposing sides of said membrane wall without breaching said membrane wall;
wherein said coupling mechanism comprises a linear actuator coupling that is disposed on said linear actuator and comprises an outer surface male connector; and
wherein said coupling mechanism comprises a scaffold structure coupling that is disposed on said scaffold structure and comprises an inner surface male connector;
wherein said membrane wall has an outer surface and an inner surface, wherein said outer surface comprises an outer female socket configured to receive said outer surface male connector and said inner surface comprises an inner female socket configured to receive said inner surface male connector.

31. The bioreactor of claim 21, wherein said coupling mechanism is permanently or temporarily attached to said membrane.

32. The bioreactor of claim 21, wherein said coupling mechanism is permanently or temporarily coupling said linear actuator to said scaffold structure.

33. The bioreactor of claim 1, wherein said bioreactor chamber is configured to be hermetically sealed.

34. The bioreactor of claim 1, wherein said bioreactor chamber is configured to be aseptically sealed.

35. The bioreactor of claim 1, wherein the inner portion of said bioreactor chamber does not contain exposed metal surfaces.

36. The bioreactor of claim 1, wherein the control system and linear actuator are disposed outside of the bioreactor chamber, and do not permeate the bioreactor chamber.

37. The bioreactor of claim 1, further comprising one or more ports disposed on said bioreactor chamber.

38. The bioreactor of claim 1, wherein said bioreactor chamber is configured to permit gas and/or nutrient exchange.

39. The bioreactor of claim 1, further comprising a removable lid assembly.

40. The bioreactor of claim 39, wherein said removable lid assembly has one or more ports for the flow of gases and/or nutrients.

41. The bioreactor of claim 39, wherein said removable lid assembly is configured to permit gas and/or nutrient exchange.

42. The bioreactor of claim 1, wherein said membrane wall has sufficient flexibility whereby said membrane wall can be displaced resultant to said linear motion in a linear direction for a distance of one the following:
a range of about 1 mm to about 10 mm;
a range of about 1 mm to about 5 mm;
a range of about 1 mm to about 6 mm;
a range of about 2 mm to about 4 mm; or
about 3 mm.

43. The bioreactor of claim 1, wherein said membrane wall has sufficient flexibility whereby said membrane wall can be displaced resultant to said linear motion in a linear direction for a distance of one the following:
a range of about 1 mm to about 10 cm;
a range of about 10 cm to about 1 m; or
a range of about 1 m to about 3 m.

44. The bioreactor of claim 21, wherein said membrane wall has sufficient flexibility in the linear direction so as to permit said linear actuator and said scaffold structure to travel with respect to one another, in response to said linear motion, causing said membrane wall to flex in an ample manner so as to allow said linear actuator and said scaffold structure to couple with one another.

45. The bioreactor of claim 1, wherein said membrane wall has sufficient elasticity in the linear direction so as to permit said linear actuator and said scaffold structure to travel with respect to one another, in response to said linear motion, causing said membrane wall to stretch in an ample manner so as to allow said linear actuator and said scaffold structure to couple with one another.

46. The bioreactor of claim 1, wherein said membrane wall has sufficient deformability in the linear direction so as to permit said linear actuator and said scaffold structure to travel with respect to one another, in response to said linear motion, causing said membrane wall to deform in an ample manner so as to allow said linear actuator and said scaffold structure to couple with one another.

47. The bioreactor of claim 21, wherein said membrane wall is configured to allow movement in the linear direction so as to permit said linear actuator and said scaffold structure to travel with respect to one another, in response to said linear motion, causing said membrane wall to move in an ample manner so as to allow said linear actuator and said scaffold structure to couple with one another.

48. The bioreactor of claim 1, further comprising a securement means for securing said scaffold structure in place.

49. The bioreactor of claim 48, wherein said securement means is a clamp or screw adjustably mounted to said chamber wherein said clamp or screw is configured to make contact with said membrane to impart a force on said membrane to be transferred to said scaffold structure for maintaining a desired position of said scaffold structure.

50. The bioreactor of claim 1, wherein said bioreactor chamber includes any one of the following structures: housing, enclosure, box, container, casing, tank, compartment, cavity, pipe, or trunk.

51. A bioreactor device, said device comprising:
a bioreactor chamber and a membrane wall disposed on said bioreactor chamber, wherein said bioreactor chamber is configured to hold a scaffold structure or other component; and
said membrane wall is configured to allow transfer of linear motion to said scaffold structure or other component without breaching said membrane wall.

52. The bioreactor device of claim 51, wherein said bioreactor chamber said membrane wall are configured to maintain sterility or sanitation within said bioreactor chamber while said bioreactor chamber is closed.

53. The device of claim 51, wherein said linear motion is a type of motion that can be generated by a linear actuator disposed outside said bioreactor chamber.

54. The device of claim 51, wherein said device is provided as part of a kit, wherein said kit includes a linear actuator, wherein said linear actuator is configured to provide said linear motion.

55. A system configured to receive said bioreactor device of claim 51, said system comprising:
a linear actuator disposed outside said bioreactor chamber, wherein said linear actuator is configured to provide said linear motion; and
a linear transfer means for transferring linear motion between said linear actuator and said scaffold structure or said other component without breaching said membrane wall.

56. The system of claim 54, further comprising:
a control system in communication with said linear actuator configured to control the movement of said linear actuator.

57. The system of claim 56, wherein said linear transfer means is comprised of a coupling mechanism, wherein said coupling mechanism is configured to couple said linear actuator to said scaffold structure, in response to said linear motion, on opposing sides of said membrane wall without breaching said membrane wall.

58. The system of claim 55, wherein said linear transfer means is comprised of a coupling mechanism, wherein said coupling mechanism is configured to couple said linear actuator to said scaffold structure, in response to said linear motion, on opposing sides of said membrane wall without breaching said membrane wall.

59. A bioreactor comprising:
a bioreactor chamber;
a membrane wall disposed on said bioreactor chamber;
a scaffold structure disposed inside said bioreactor chamber;
a linear actuator disposed outside said bioreactor chamber;
a linear transfer means for transferring linear motion between said linear actuator and said scaffold structure without breaching said membrane wall; and
a control system in communication with said linear actuator configured to control the movement of said linear actuator.

60. The bioreactor of claim 59, wherein said bioreactor chamber and said membrane wall are configured to separate from said linear actuator, said linear transfer means, and said control system.

61. The bioreactor of claim 60, wherein said bioreactor chamber and membrane wall are configured to maintain sterility or sanitation within said bioreactor chamber while said bioreactor is closed.

62. The bioreactor of claim 59, wherein said bioreactor chamber and said membrane wall is configured to maintain sterility or sanitation within said bioreactor chamber while said bioreactor is closed.

63. The bioreactor of claim 59, wherein said linear transfer means is comprised of a coupling mechanism, wherein said coupling mechanism is configured to couple said linear actuator to said scaffold structure, in response to said linear motion, on opposing sides of said membrane wall without breaching said membrane wall.

64. A bioreactor comprising:
a bioreactor chamber;
a membrane wall disposed on said bioreactor chamber, wherein said bioreactor chamber and said membrane wall are configured to maintain sterility or sanitation within said bioreactor chamber while said bioreactor chamber is closed;
a scaffold structure disposed inside said bioreactor chamber;
an actuator disposed outside said bioreactor chamber;
a transfer means for transferring motion between said actuator and said scaffold structure without breaching said membrane wall; and
a control system in communication with said actuator configured to control the movement of said actuator.
